SEQUENCE LISTING (2 sequences)

SEQ ID NO: 1 - RNA, 22 nt, Homo sapiens
  source 1..22: mol_type = other RNA
tggcagtgtattgttagctggt

SEQ ID NO: 2 - RNA, 91 nt, Homo sapiens
  source 1..91: mol_type = other RNA
ctgtgtgtgatgagctggcagtgtattgttagctggttgaatatgtgaatggcatcggctaacatgcaactgctgtcttattgcatataca

(19) United States
(12) Patent Application Publication
(10) Pub. No.: US 2025/0376681 A1
(43) Pub. Date: Dec. 11, 2025

(54) MIR449A AS A THERAPEUTIC FOR NEURODEGENERATIVE DISORDERS

(71) Applicant: NATIONAL INSTITUTE OF IMMUNOLOGY, Delhi (IN)

(72) Inventors: Pushkar SHARMA, Delhi (IN); Monika CHAUHAN, Delhi (IN)

(21) Appl. No.: 18/878,560

(22) PCT Filed: Sep. 15, 2023
(86) PCT No.: PCT/IN2023/050861

§ 371 Date: Dec. 23, 2024

(30) Foreign Application Priority Data
Sep. 16, 2022 (IN) 202211052962

(51) Int. Cl. C12N 15/113

(52) U.S. Cl. CPC C12N 15/113; C12N 2310/141

(57) ABSTRACT

The present disclosure provides a method for expressing miR-449a in a neuronal cell by administering a viral vector comprising a nucleotide sequence containing a miRNA-449a sequence. The present disclosure also provides a method for treating a cognitive impairment due to miR-449a dysregulation in a patient by administering a viral vector comprising a nucleotide sequence containing a miRNA-449a sequence to the patient. The viral vector decreases the levels of cell cycle proteins such as CDC25A and cyclin D1 proteins and decreases the levels of proliferating cell nuclear antigen (PCNA) and cleaved caspase-3 in the patients. The methods of the present disclosure reduce the neurodegeneration and improve the cognitive and functional decline in AD patients.

### CROSS-REFERENCE TO RELATED APPLICATIONS

The present application is a filing under 35 U.S.C. 371 as the National Stage of International Application No. PCT/IN2023/050861, filed Sep. 15, 2023, entitled “MIR449A AS A THERAPEUTIC FOR NEURODEGENERATIVE DISORDERS,” which claims priority to Indian Application No. 202211052962 filed with the Intellectual Property Office of India on Sep. 16, 2022, both of which are incorporated herein by reference in their entirety for all purposes.

### INCORPORATION BY REFERENCE OF MATERIAL IN XML FILE

This application incorporates by reference the Sequence Listing contained in the following XML file being submitted concurrently herewith:

File name: 4863-01900 FP11717 Seq Listing MiR449a_edited.xml, created on Jan. 7, 2025; and having a file size of 7.00 KB. The information in the Sequence Listing is incorporated herein in its entirety for all purposes.

### FIELD OF THE DISCLOSURE

The present invention relates to methods of expressing miR-449a in a neuronal cell by employing a viral vector comprising a nucleotide sequence containing a miRNA-449a sequence. The present invention also relates to methods for treating a mild cognitive impairment (MCI) in a patient. The present invention further relates to the methods and compositions for treating a cognitive impairment due to miR-449a dysregulation by administering to the patient a viral vector comprising a nucleotide sequence containing a miRNA-449a sequence.

### BACKGROUND OF THE DISCLOSURE

Neural precursor cells differentiate into neurons by exiting the cell cycle (Galderisi, Jori et al. 2003) and the cell cycle of terminally differentiated neurons remains arrested for the remainder of their life span (Dehay and Kennedy 2007). Therefore, the machinery involved in cell cycle progression, which constitutes of key regulatory elements like cyclins, cyclin dependent kinases (CDKs) and CDK inhibitors, needs tight regulation (Frade and Ovejero-Benito 2015). These and other elements form a network that works in an orchestrated way to promote the progression of the cell cycle. For instance, the expression of D-type cyclins like cyclin D1 is induced by signaling pathways stimulated by mitogenic signals (Choi and Anders 2014). Cyclin D associates with CDK4/6 and activates it, which in turn phosphorylates retinoblastoma (Rb) or other related proteins. In its unphosphorylated state, Rb proteins keep the transcription factor E2F sequestered and Rb-phosphorylation promotes dissociation of E2F and transcriptional activation. E2F is involved in transcription of S-phase cyclin E, which activates CDK2 to promote S-phase progression (Topacio, Zatulovskiy et al. 2019). CDKs are regulated by reversible phosphorylation; CAK phosphorylation of the activation loop of CDKs (at T160 in the case of CDK1) activates them whereas phosphorylation by Myt1 and Wee1 inhibits their activity. Protein phosphatase CDC25 and its isoforms dephosphorylate the Myt1 and Wee1 phosphorylated sites (T14 and Y15 in CDK1) and prevent activation of CDKs (Lew and Kornbluth 1996). Rb knockout causes aberrant cell cycle reentry via E2F-1 (Andrusiak, Vandenbosch et al. 2012) suggesting that it is critical for neuronal cell cycle to remain suppressed.

There is substantial evidence suggesting that neurons can re-enter the cell cycle and undergo DNA replication in response to neurotoxic insults like DNA-damage and Aβ-42 amyloid peptide (Thornton, Vink et al. 2006, Varvel, Bhaskar et al. 2008) (Suram, Hegde et al. 2007). However, aberrant cell cycle re-entry does not culminate in mitosis, instead, it results in neuronal apoptosis and neuronal loss in the cortex (Folch, Junyent et al. 2012). The expression and activity of cell cycle regulators like the ones mentioned above is modulated, which contributes to the process of cell cycle related neuronal apoptosis (CRNA) (Herrup and Busser 1995, Park, Obeidat et al. 2000, Lee, Casadesus et al. 2009). Aβ-42 generated during Alzheimer's disease (AD) is known to cause aberrant cell cycle re-entry of cortical neurons, a commonly observed phenomenon in AD animal models (Varvel, Bhaskar et al. 2008, Li, Cheung et al. 2011) and in the brains of AD patients (Giovanni, Wirtz-Brugger et al. 1999, Yang, Geldmacher et al. 2001, Yang, Mufson et al. 2003, Crews and Masliah 2010).

miRNA are ˜22 nucleotide small non-coding RNAs that function by typically binding to the 3′-untranslated region (3′-UTR) of the target mRNA (Ha and Kim 2014) and regulate the expression of the target by facilitating mRNA degradation or its translational repression. miRNA can regulate neuronal proliferation, differentiation as well as apoptosis (Bartel 2004, Nohata, Sone et al. 2011, Fabian and Sonenberg 2012, Son, Ka et al. 2014, Zhang, Tan et al. 2019). While independent studies have indicated that several miRNA clusters are involved in the regulation of cell cycle related genes (Otto, Candido et al. 2017, Mens and Ghanbari 2018), it still remains relatively unclear if they contribute to neuronal differentiation.

MicroRNA shave been identified to be dysregulated upon generation of Aβ42 in Alzheimer's disease, thereby inducing CRNA. The present invention uses viral vectors to express miR-449a to prevent aberrant cell cycle re-entry induced by Aβ42 and prevent neuronal apoptosis. The present disclosure provides methods for treating a cognitive impairment in patients caused by miR-449a dysregulations by administering a viral vector to express miR-449a targeting the cell cycle proteins CDC25A and cyclin D1. Thus, the present invention provides a solution for mitigation of CRNA that may contribute to a neuronal loss.

### SUMMARY OF THE DISCLOSURE

The present disclosure provides a method for expressing miR-449a in a neuronal cell, comprising introducing into the cell a viral vector comprising a nucleotide sequence containing a miRNA-449a sequence.

The present disclosure provides a method for treating the cognitive impairment due to miR-449adysregulation in a patient in need thereof, comprising administering to the patient a viral vector comprising a nucleotide sequence containing a miRNA-449a sequence.

The present disclosure provides a method for reducing neurodegeneration in a patient suffering from cognitive impairment or at a risk of developing AD, comprising administering to the patient a viral vector having a nucleotide sequence containing a miRNA-449a sequence.

The present disclosure provides a method for treating a mild cognitive impairment (MCI) in a patient, comprising administering to the patienta viral vector comprising a nucleotide sequence containing a miRNA-449a sequence.

The present disclosure also provides a method for treating a pre-clinical stage cognitive impairment in a patient, comprising administering to the patient a viral vector comprising a nucleotide sequence containing a miRNA-449a sequence.

The present disclosure provides a composition for use in treating a cognitive impairment due to miRNA-449a dysregulation, wherein the composition comprises a viral vector comprising a nucleotide sequence containing a miRNA-449a sequence.

The present disclosure provides use of a viral vector comprising a nucleotide sequence containing a miRNA-449a sequence for treating various clinical stages of cognitive impairment due to miRNA-449a dysregulation as described herein.

The present disclosure provides a viral vector comprising a nucleotide sequence containing a miRNA-449a sequence for use as a medicament for treatment of various clinical stages of AD as described herein.

In some embodiments, the nucleotide sequence containing a miRNA-449a sequence in the viral vector comprises a miRNA-449a hairpin loop sequence including a mature miRNA-449a sequence.

### BRIEF DESCRIPTION OF THE DRAWINGS

FIG. 1A shows a fold change with respect to the levels of miR-449a expression in neurons derived from rat embryo cortex that were allowed to differentiate for 2, 5 and 7 days in vitro (DIV).

FIG. 1B shows a Western Blot of the levels of actin, PCNA, cyclin D1 and cleaved caspase 3 in the rat cortical neurons transfected with antagomir for miR-449a or a control antagomir.

FIG. 1C shows the quantification of the levels of PCNA from the Western blot of FIG. 1B.

FIG. 1D shows the quantification of the levels of cleaved caspase 3 from the Western blot of FIG. 1B.

FIG. 1E shows microscope images of immunofluorescence and TUNEL assays performed to detect BrdU incorporation (red) or apoptosis (green) on rat cortical neurons transfected with anti-miR-449a or a control.

FIG. 1F shows the quantification of the percent cell number for the BrdU⁺, TUNEL⁺ and BrdU⁺/TUNEL⁺ in FIG. 1E (Data presented as mean±SEM, *p<0.05, t-test, N=3).

FIG. 2A shows fold change with respect to the levels of miR-449a in ratcortical neurons untreated or treated with Aβ42 for 48h. (Data presented as mean±SEM, **p<0.01, t-test, N=3)

FIG. 2B shows fold change with respect to the levels of miR-449a incortical neurons from wild type (WT) or TgAD (Tg) mice. (Data presented as Mean±SEM, ****p<0.0001, t-test, N=8; **p<0.0001)

FIG. 2C shows fold change with respect to the levels of miR-449a in the RNA isolated from frontal cortex tissue of two AD patients or age matched healthy controls. (Data presented as Mean±SEM, n=3 of two biological samples, *p<0.05, **p<0.01, t-test, N=2)

FIG. 2D shows a Western Blot for the rat cortical neurons transduced with lentivirus expressing miR-449a or control lentivirus (pLKO) followed by treatment with Aβ₄₂ for 48 h.

FIG. 2E shows the quantification of the levels of PCNA and cleaved caspase 3 from the Western blot of FIG. 2D. (Data presented as mean±SEM, *p<0.05, ANOVA, N=3)

FIG. 2F shows a Western Blot for the cortical neurons cultured from WT or TgAD mice transduced with miR-449a or control lentivirus (pLKO) for 48h.

FIG. 2G shows the quantification of the levels of PCNA and cleaved caspase 3 from the Western blot of FIG. 2F. (Data presented as mean±SEM, *p<0.05 by ANOVA, N=3)

FIG. 2H shows microscope images of immunofluorescence and TUNEL assays performed to detect BrdU incorporation or apoptosis on cortical neurons of mouse transduced with lentivirus expressing miR-449a or control virus (pLKO) and treated with Aβ₄₂ for 48h, followed by incubation with BrdU.

FIG. 2I shows the quantification of the percent cell number for the BrdU⁺ (red), TUNEL⁺ (green) and BrdU⁺/TUNEL⁺ (yellow) in FIG. 1E (Data presented as mean±SEM, *p<0.05, t-test, N=3).

FIG. 3A shows a Venn diagram showing seven common targets predicted with miRDB, Target Scan and RNA22.

FIG. 3B shows the putative targets relevant for the cell cycle derived from Venn diagram of FIG. 3A.

FIG. 3C shows the fold change in the levels of CDC25A mRNA transcript when the rat cortical neurons were transfected with anti-miR-449a or a control.

FIG. 3D shows the fold change in the levels of cyclin D1 mRNA transcript when the rat cortical neurons were transfected with anti-miR-449a or a control.

FIG. 3E shows a Western Blot for the rat cortical neuronstransduced with miR-449a or control lentivirus, treated or left untreated with Aβ₄₂.

FIG. 3F shows the quantification of the levels of CDC25A mRNA transcription neurons transduced with miR-449a or control lentivirus, treated or left untreated with Aβ₄₂. (Data presented as mean±SEM, *p<0.05, ANOVA, N=3)

FIG. 3G shows the quantification of the levels of CDC25A protein from the Western blot of FIG. 3E in neurons transduced with miR-449a or control lentivirus, treated or left untreated with Aβ₄₂. (Data presented as mean±SEM, *p<0.05, ANOVA, N=3)

FIG. 3H shows a Western Blot of cyclin D1 for the rat cortical neurons transduced with miR-449a or control lentivirus, treated or left untreated with Aβ₄₂.

FIG. 3I shows the quantification of the levels of cyclin D1 mRNA transcriptin neurons transduced with miR-449a or control lentivirus, treated or left untreated with Aβ₄₂. (Data presented as mean±SEM, *p<0.05, ANOVA, N=3)

FIG. 3J shows the quantification of the levels of cyclin D1 from the Western blot of FIG. 3H in neurons transduced with miR-449a or control lentivirus, treated or left untreated with Aβ₄₂. (Data presented as mean±SEM, *p<0.05, ANOVA, N=3)

FIG. 3K shows a Western Blot for the cortical neurons cultured from WT or TgAD mice transduced with miR-449a or control lentivirus for 48 h. The neurons were also infected with adenovirus expressing CDC25A or GFP (control).

FIG. 3L shows the quantification of the levels of PCNA which were normalized with respect to actin from the Western blot of FIG. 3K with respect to pLKO transduced neurons. (Data presented as mean±SEM of at least three replicates, *p<0.05 with paired t-test.)

FIG. 3M shows the quantification of the levels of cleaved caspase 3 which were normalized with respect to actin from the Western blot of FIG. 3K with respect to pLKO transduced neurons. (Data presented as mean±SEM of at least three replicates, *p<0.05 with paired t-test.)

FIG. 4A shows a fold change in luciferase activity in rat cortical neurons transfected with a luciferase reporter plasmid containing CDC25A 3′-UTR and transduced with miR-449a or pLKO (control) lentivirus and treated with Aβ₄₂ for 48h. (Data presented as mean±SEM, ANOVA, *p<0.05, N=3.)

FIG. 4B shows the corresponding binding site in mutant UTR where the miR-449a binds to CDC25A (Indicated in red).

FIG. 4C shows a fold change in the luciferase activity in rat cortical neurons transfected with a luciferase reporter plasmid containing cyclin D1 3′-UTR and transduced with miR-449a or pLKO (control) lentivirus and treated with Aβ₄₂ for 48h. (Data presented as mean±SEM, ANOVA, *p<0.05, N=3.)

FIG. 4D shows the corresponding binding site in mutant UTR where the miR-449a binds to cyclin D1 (Indicated in red).

FIG. 4E shows a Western Blot of CDC25A from the rat cortical neurons transfected with anti-miR-449a or control antagomir. The neurons were also infected with adenovirus expressing CDC25A or GFP (control).

FIG. 4F shows quantification of the levels of CDC25A from the Western blot of FIG. 4E. (Data presented as mean±SEM, t-test, *p<0.05, N=3)

FIG. 4G shows a Western Blot of cyclin D1 from the rat cortical neurons transfected with anti-miR-449a or control antagomir. The neurons were also infected with adenovirus expressing CDC25A or GFP (control).

FIG. 4H shows quantification of the levels of cyclin D1 from the Western blot of FIG. 4G. (Data presented as mean±SEM, t-test, *p<0.05, N=3)

FIG. 4I shows a Western Blot of CDC25A from cortical neurons from WT/TgAD mice transduced with miR-449a or control lentivirus.

FIG. 4J shows a quantification of the levels of CDC25A from the Western blot of FIG. 4I. (Data presented as mean±SEM, ANOVA, *p<0.05, N=3)

FIG. 4K shows a Western Blot of Cyclin D1 from cortical neurons from WT/TgAD mice transduced with miR-449a or control lentivirus.

FIG. 4L shows a quantification of the levels of cyclin D1 from the Western blot of FIG. 4K. (Data presented as mean±SEM, ANOVA, *p<0.05, N=3)

FIG. 4M shows a Western Blot for the rat cortical neurons treated or left untreated with Aβ₄₂ for 48 h were transduced with miR-449a or control lentivirus. The neurons were also infected with adenovirus expressing CDC25A or GFP (control).

FIG. 4N shows quantification of the levels of PCNA and cleaved caspase 3 which were normalized with respect to actin from the Western blot of FIG. 4M. (Data presented as mean±SEM, ANOVA, *p<0.05, N=3)

FIG. 4O shows Western Blot for the rat cortical neurons treated or left untreated with Aβ₄₂ for 48h were transduced with miR-449a or control lentivirus. The neurons were also infected with adenovirus expressing cyclin D1 or GFP (control).

FIG. 4P shows quantification of the levels of PCNA and cleaved caspase 3 which were normalized with respect to actin from the Western blot of FIG. 4M. (Data presented as mean±SEM, ANOVA, *p<0.05, **p<0.01, ***p<0.001, N=3)

FIG. 5A shows a schematic protocol of lentivirus injection to 6-month-old to female WT and TgAD mice injected stereotaxically in the frontal cortex with Lv-miR-449a or Lv-NC (negative control).

FIG. 5B shows the results of the Morris water maze test performed with a hidden probe to determine the mean escape latency to reach the platform at indicated time.

FIG. 5C shows the results of latency to reach the platform on the final day (Day 4) of training in Lv-miR449a injected TgAD mice. (Data presented as mean±SEM, N=5-8 mice/group; *p<0.05; **p<0.01 by unpaired t-test).

FIG. 5D shows representative track plots of 2 mice from each group in MWM test described in FIG. 5C.

FIG. 5E shows results Morris water maze test of the Probe trial performed 24 hours after the last hidden platform test in the Morris water maze (Fig. B). The number of platform area crossings in the target quadrant is also shown. (Data represented as mean±SEM, N=5-8 mice/group; *p<0.05; **p<0.01, t-test.)

FIG. 5F shows for Y-maze test for mice acclimatized in the maze with one arm closed for 3 min and recorded the alternations and % spontaneous alternation for each mouse. (Data represented as mean±SEM, N=5-8 mice/group, *p<0.05, t-test.)

FIG. 6A shows results for average swimming speed (m/s) of mice from each group in Morris water maze probe test (FIG. 5E). (Data represented as mean±SEM, N=5-8 animals from each cohort, ns—not significant by ANOVA.)

FIG. 6B shows number of total arm entries in Y maze (FIG. 5F) is shown. (Data represented as mean±SEM, N=6-8 animals from each cohort, not significant by ANOVA.)

FIG. 7A shows representative images of IFA of cortex of WT and TgAD mouse for PCNA (S phase) and NeuN (mature neurons) along with TUNEL labeling (apoptosis).

FIG. 7B shows the Fold change in TUNEL⁺/PCNA⁺/NeuN⁺ cells with respect to uninjected WT mice. (Data presented as mean±SEM of N=3 mice per group, n=3 sections per mice, *p<0.05 by ANOVA.)

FIG. 7C shows representative images of the frontal cortex region of the immunohistochemistry of the brain sections of lentivirus injected mice for the expression of GFP (expressed from pLKO vector).

FIG. 7D shows the percentage of GFP positive neurons from the immunohistochemistry data from FIG. 7C, scale bar 100 μm. (Data represented as mean±SEM of N=3 mice per group, n=3 sections per mice, not significant by ANOVA.)

### BRIEF DESCRIPTION OF THE SEQUENCE LISTING

SEQ ID NO. 1 is the nucleic acid sequence of a mature human miR-449a:

UGGCAGUGUAUUGUUAGCUGGU

SEQ ID NO. 2 is the nucleic acid sequence of human miR-449a hairpin loop sequence used in the viral vector:

CUGUGUGUGAUGAGCUGGCAGUGUAUUGUUAGCUGGUUGAAUAUGUGAA
UGGCAUCGGCUAACAUGCAACUGCUGUCUUAUUGCAUAUACA

SEQ ID NO. 3 is the nucleic acid sequence of murine CDC25A UTR:

UCAUUAAGUCAAAAUACUGCCAU

SEQ ID NO. 5 is the nucleic acid sequence of murine Cyclin D1 UTR:

GUUUUACMUGUCAGACUGCCAU

SEQ ID NO. 6 is the nucleic acid sequence of Cyclin D1 mutant UTR:

GUUUUACUUTGUCAGGAGGAUU

SEQ ID NO. 7 is the nucleic acid sequence of mature murine miR-449a:

UGGUCGAUUGUUAUGUGACGGU

### DETAILED DESCRIPTION OF THE DISCLOSURE

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The use of the expression “at least” or “at least one” suggests the use of one or more elements or ingredients or quantities, as the use may be in the embodiment of the disclosure to achieve one or more of the desired objects or results. Throughout this specification, the word “comprise”, or variations such as “comprises” or “comprising” or “containing” or “has” or “having”, or “including but not limited to” wherever used, will be understood to imply the inclusion of a stated element, integer or step, or group of elements, integers or steps, but not the exclusion of any other element, integer or step, or group of elements, integers or steps.

Reference throughout this specification to “one embodiment”, “an embodiment”, or “some embodiments” means that a particular feature, structure or characteristic described in connection with the embodiment may be included in at least one embodiment of the present disclosure. Thus, the appearances of the phrases “in one embodiment”, “in an embodiment”, or “in some embodiments” in various places throughout this specification may not necessarily all refer to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

The term “subject” or “patient” as used herein refers to any mammal including, without limitation, humans and other primates (e.g., chimpanzees and other apes and monkey species), farm animals (e.g., cattle, sheep, pigs, goats and horses), domestic mammals (e.g., dogs and cats), and laboratory animals (e.g., rodents such as mice, rats, and guinea pigs). In some embodiments, the patient is a mammal. In some embodiments, the patient is a human.

MicroRNA such as miR-34a, miR-17-5p, miR-15, miR-449a, have been reported previously to be implicated in the process of cell cycle regulation (Bueno and Malumbres 2011) and in addition several of these also perform important neuronal functions such as neuronal stem cell differentiation (Aranha, Santos et al. 2011) and neurite outgrowth (Agostini, Tucci et al. 2011). While altered expression of some of these cell cycle related miRNA has been reported in neurodegenerative disorders (Bushati and Cohen 2008, Junn and Mouradian 2012, Zhang, Cheng et al. 2014, Marcuzzo, Bonanno et al. 2015), their correlation with the neuronal cell cycle regulation and neuronal loss has remained unknown. RNA sequencing revealed the identity of several miRNA that exhibited significantly altered expression in cortical neurons of a model for Alzheimer's Disease (AD), APP/PS1 (TgAD). The role of miR-449a in CRNA was studied in detail. miR-449a belongs to miR-449 cluster (Kochegarov, Moses et al. 2013) which is located in the second intron of CDC20B gene on chromosome 5. It is highly expressed in the mucocilliary epithelia of lungs (Lize, Herr et al. 2010, Song, Walentek et al. 2014). In the brain, miR-449a is expressed during the proliferative phase of embryonic neurogenesis (Barca-Mayo and De Pietri Tonelli 2014) and in adult brain choroid plexus (Redshaw, Wheeler et al. 2009). It is also essential for the production of intermediate progenitors during cortical development (Wu, Bao et al. 2014, Fededa, Esk et al. 2016). The tumor suppressor role of miR-449a has been extensively studied in dividing cells in various types of cancers (Noonan, Place et al. 2009, Noonan, Place et al. 2010, Chen, Liu et al. 2015, Yao, Ma et al. 2015, Zhao, Ma et al. 2015, Liu, Liu et al. 2016). Previous studies have also reported that miR-449a can arrest cells in G0/G1 phase in cancerous cells thus inhibiting their growth and viability (Noonan, Place et al. 2010). The inventors have found that miR-449a can be used as a therapeutic agent to prevent aberrant cell cycle re-entry induced by Aβ₄₂ and prevent neuronal apoptosis. The cell cycle proteins CDC25A and cyclin D1 are identified as miR-449a targets, which are suppressed using miR-449a to prevent CRNA. Since CRNA may contribute to a neuronal loss, its mitigation is a possible avenue for therapeutic intervention. Thus, the regulation of miR-449a is able to reduce CRNA and thereby rescue short- and long-term memory defects and improves memory and cognitive function in a mouse model of AD.

In some embodiments, the present disclosure provides a method for expressing miR-449a in a neuronal cell, comprising introducing into the cell a viral vector comprising a nucleotide sequence containing a miRNA-449a sequence.

In some embodiments, the present disclosure provides a method for treating a cognitive impairment due to miR-449a dysregulation in a patient, comprising administering to the patient a viral vector comprising a nucleotide sequence containing a miRNA-449a sequence.

In some embodiments, the nucleotide sequence containing a miRNA-449a sequence present in the viral vector comprises a miRNA-449a hairpin loop sequence including mature miR-449a sequence.

In some embodiments, the nucleotide sequence of mature miRNA-449a comprises the following sequence: UGGCAGUGUAUUGUUAGCUGGU (SEQ ID NO. 1).

In some embodiments, the nucleotide sequence of miRNA-449a hairpin loop sequence comprises the following sequence:

(SEQ ID NO. 2)
CUGUGUGUGAUGAGCUGGCAGUGUAUUGUUAGCUGGUUGAAUAUGUGAA
UGGCAUCGGCUAACAUGCAACUGCUGUCUUAUUGCAUAUACA.

In some embodiments, the viral vector is a lentivirus vector comprising a nucleotide sequence containing a miRNA-449a sequence wherein the miRNA-449a sequence comprises a miRNA-449a hairpin loop sequence including mature miR-449a sequence.

In some embodiments, the viral vector is administered in the form of an injection or an infusion. In some embodiments, the viral vector is administrated intramuscularly, intravenously, intrathecally, intraparenchymally or intracerebroventricularly in the patient.

In some embodiments, the methods described herein for expressing miR-449a in a neuronal cell are in vivo or in vitro methods.

In some embodiments, the administration of the viral vector decreases the levels of cell cycle proteins such as cyclin D1 and CDC25A proteins in neuronal cells of the patient.

In some embodiments, the introduction of the viral vector comprising a nucleotide sequence of miR-449a into the neuronal cell decreases the levels of CDC25A protein in the neuronal cell. Accordingly, in some embodiments, provided herein is a method of decreasing the levels of CDC25A protein in a neuronal cell comprising introducing into the cell a viral vector expressing the miRNA-449a described herein. In some embodiments, the viral vector decreases the levels of CDC25A protein by about 2 to about 6-fold, including values and ranges thereof, compared to levels of CDC25A protein prior to introduction of the viral vector or compared to levels of CDC25A protein in cells transduced with a control vector. In some embodiments, the viral vector decreases the levels of CDC25A protein in neuronal cells by about 2 to about 6-fold, 2 to about 5.5-fold, about 2 to about 5-fold, 2 to about 4.5-fold, about 2 to about 4-fold, about 2 to about 3.5-fold, about 2 to about 3-fold, about 2 to about 2.5-fold, 2.5 to about 6-fold, 2.5 to about 5.5-fold, about 2.5 to about 5-fold, 2.5 to about 4.5-fold, about 2.5 to about 4-fold, about 2.5 to about 3.5-fold, about 2.5 to about 3-fold, 3 to about 6-fold, 3 to about 5.5-fold, about 3 to about 5-fold, 3 to about 4.5-fold, about 3 to about 4-fold, 3 to about 3.5-fold, 3.5 to about 6-fold, 3.5 to about 5.5-fold, about 3.5 to about 5-fold, 3.5 to about 4.5-fold, about 3.5 to about 4-fold, 4 to about 6-fold, 4 to about 5.5-fold, about 4 to about 5-fold, 4 to about 4.5-fold, 4.5 to about 6-fold, 4.5 to about 5.5-fold, about 4.5 to about 5-fold, 5 to about 6-fold, 5 to about 5.5-fold, about 5.5 to about 6-fold, including values and ranges thereof, compared to levels of CDC25A protein prior to introduction of the viral vector or compared to levels of CDC25A in cells transduced with a control vector. In some embodiments, the introduction of the viral vector decreases the levels of CDC25A protein in neuronal cells by about 2-fold, 2.3-fold, 2.4-fold, 2.5-fold, 2.8-fold, 3-fold, 3.2-fold, 3.4-fold, 3.5-fold, 3.7-fold, 4-fold, 4.5-fold, 5-fold, 5.5-fold or by about 6-fold, compared to levels of CDC25A protein prior to administration of the viral vector.

In some embodiments, the introduction of the viral vector comprising a nucleotide sequence of miR-449a into the neuronal cell decreases the levels of CDC25A protein in neuronal cells by about 30-80%, including values and ranges thereof, compared to levels of CDC25A protein prior to introduction of the viral vector or compared to levels of CDC25A in cells transduced with a control vector. In some embodiments, the introduction of the viral vector decreases the levels of CDC25A protein in neuronal cells by about 30-80%, 30-75%, 30-70%, 30-65%, 30-60%, 30-55%, 30-50%, 30-45%, 35-80%, 35-75%, 35-70%, 35-65%, 35-60%, 35-55%, 35-50%, 40-80%, 40-75%, 40-70%, 40-65%, 40-60%, 40-50%, 50-80%, 50-75%, 50-70%, 50-65%, 50-60%, 60-80%, 60-75%, 60-70%, 65-80%, or 70-80%, including values and ranges thereof, compared to levels of CDC25A protein prior to introduction of the viral vector or compared to levels of CDC25A in cells transduced with a control vector. In some embodiments, the introduction of the viral vector decreases the levels of CDC25A protein in neuronal cells by about 30%, 35%, 40%, 45%, 50%, 55%, 60%, 65%, 70%, 75%, or about 80%, including values and ranges thereof, compared to the levels of CDC25A protein prior to introduction of the viral vector or compared to levels of CDC25A in cells transduced with a control vector.

In some embodiments, the introduction of the viral vector comprising a nucleotide sequence of miR-449a into the neuronal cell decreases the levels of cyclin D1 protein in neuronal cells of the patient, compared to levels of cyclin D1 protein prior to introduction of the viral vector or compared to levels of cyclin D1 in cells transduced with a control vector. Accordingly, in some embodiments, provided herein is a method of decreasing the levels of cyclin D1 protein in neuronal cells comprising introducing into the neuronal cell a viral vector comprising a nucleotide sequence of miR-449a as described herein. In some embodiments, the introduction of the viral vector into the neuronal cell decreases the levels of cyclin D1 protein by about 2 to about 8-fold, including values and ranges thereof, compared to levels of cyclin D1 protein prior to introduction of the viral vector or compared to levels of cyclin D1 protein in cells transduced with a control vector. In some embodiments, the introduction of the viral vector decreases levels of cyclin D1 protein in neuronal cells by about 2 to about 8-fold, 2 to about 7.5-fold, 2 to about 7-fold, 2 to about 6.5-fold, 2 to about 6-fold, 2 to about 5.5-fold, about 2 to about 5-fold, 2 to about 4.5-fold, about 2 to about 4-fold, about 2 to about 3.5-fold, about 2 to about 3-fold, about 2 to about 2.5-fold, 2.5 to about 8-fold, 2.5 to about 7.5-fold, 2.5 to about 7-fold, 2.5 to about 6.5-fold, 2.5 to about 6-fold, 2.5 to about 5.5-fold, about 2.5 to about 5-fold, 2.5 to about 4.5-fold, about 2.5 to about 4-fold, about 2.5 to about 3.5-fold, about 2.5 to about 3-fold, 3 to about 8-fold, 3 to about 7.5-fold, 3 to about 7-fold, 3 to about 6.5-fold, 3 to about 6-fold, 3 to about 5.5-fold, about 3 to about 5-fold, 3 to about 4.5-fold, about 3 to about 4-fold, 3 to about 3.5-fold, 3.5 to about 8-fold, 3.5 to about 7.5-fold, 3.5 to about 7-fold, 3.5 to about 6.5-fold, 3.5 to about 6-fold, 3.5 to about 5.5-fold, about 3.5 to about 5-fold, 3.5 to about 4.5-fold, about 3.5 to about 4-fold, 4 to about 8-fold, 4 to about 7.5-fold, 4 to about 7-fold, 4 to about 6.5-fold, 4 to about 8-fold, 4 to about 7.5-fold, 4 to about 7-fold, 4 to about 6.5-fold, 4 to about 6-fold, 4 to about 5.5-fold, about 4 to about 5-fold, 4 to about 4.5-fold, 4.5 to about 8-fold, 4.5 to about 7.5-fold, 4.5 to about 7-fold, 4.5 to about 6.5-fold, 4.5 to about 6-fold, 4.5 to about 5.5-fold, about 4.5 to about 5-fold, 5 to about 8-fold, 5 to about 7.5-fold, 5 to about 7-fold, 5 to about 6.5-fold, 5 to about 6-fold, 5 to about 5.5-fold, about 5.5 to about 6-fold, 6 to about 8-fold, 6 to about 7.5-fold, 6 to about 7-fold, 6 to about 6.5-fold, 6.5 to about 8-fold, 6.5 to about 7.5-fold, 6.5 to about 7-fold, 7 to about 8-fold, 7 to about 7.5-fold, including values and ranges thereof, compared to levels of cyclin D1 protein prior to introduction of the viral vector or compared to levels of cyclin D1 protein in cells transduced with a control vector. In some embodiments, the administration of the viral vector decreases the levels of cyclin D1 protein in neuronal cells by about 2-fold, 2.3-fold, 2.4-fold, 2.5-fold, 2.8-fold, 3-fold, 3.2-fold, 3.4-fold, 3.5-fold, 3.7-fold, 4-fold, 4.5-fold, 5-fold, 5.5-fold, by about 6-fold, by about 6.5-fold, by about 7-fold, by about 7.5-fold or by about 8-fold, compared to levels of cyclin D1 protein prior to introduction of the viral vector or compared to levels of cyclin D1 protein in cells transduced with a control vector.

In some embodiments, the introduction of the viral vector comprising a nucleotide sequence of miR-449a into the neuronal cell decreases the levels of cyclin D1 protein in neuronal cells of the patient by about 20-90%, including values and ranges thereof, compared to levels of cyclin D1 protein prior to introduction of the viral vector or compared to levels of cyclin D1 in cells transduced with a control vector. In some embodiments, the introduction of the viral vector decreases levels of cyclin D1 protein in neuronal cells by about 20-90%, 20-85%, 20-80%, 20-75%, 20-70%, 20-65%, 20-60%, 20-55%, 20-50%, 20-45%, 20-40%, 20-30%, 25-90%, 25-85%, 25-80%, 25-75%, 25-70%, 25-65%, 25-60%, 25-55%, 25-50%, 25-45%, 25-40%, 25-35%, 30-90%, 30-85%, 30-80%, 30-75%, 30-70%, 30-65%, 30-60%, 30-55%, 30-50%, 30-45%, 35-90%, 35-85%, 35-80%, 35-75%, 35-70%, 35-65%, 35-60%, 35-55%, 35-50%, 40-90%, 40-85%, 40-80%, 40-75%, 40-70%, 40-65%, 40-60%, 40-50%, 50-90%, 50-95%, 50-80%, 50-75%, 50-70%, 50-65%, 50-60%, 60-90%, 60-85%, 60-80%, 60-75%, 60-70%, 65-90%, 65-85%, 65-80%, 70-90%, 70-85%, 70-80%, 80-90% including values and ranges thereof, compared to levels of cyclin D1 protein prior to introduction of the viral vector or compared to levels of cyclin D1 protein in cells transduced with a control vector. In some embodiments, the introduction of the viral vector decreases levels of cyclin D1 protein in neuronal cells by about 20%, 25%, 30%, 35%, 40%, 45%, 50%, 55%, 60%, 65%, 70%, 75%, 80%, 85% or about 90%, including values and ranges thereof, compared to levels of cyclin D1 protein prior to introduction of the viral vector or compared to levels of cyclin D1 in cells transduced with a control vector.

In some embodiments, the introduction of the viral vector comprising a nucleotide sequence of miR-449a into the neuronal cell inhibits cell cycle related neuronal apoptosis (CRNA) in the cell. Accordingly, in some embodiments, the present disclosure provides a method for inhibiting CRNA in a neuronal cell comprising introducing into the cell a viral vector comprising a nucleotide sequence of miR-449a. In some embodiments, the levels of proliferating cell nuclear antigen (PCNA) or the levels of cleaved caspase 3 in neuronal cells are employed to measure the extent of CRNA.

In some embodiments, the introduction of the viral vector into the neuronal cell inhibits CRNA in the neuronal cells of the patient by about 4 to about 12-fold, including values and ranges thereof, as measured by a decrease in levels of PCNA when compared to levels of PCNA prior to introduction of the viral vector or compared to levels of PCNA in cells transduced with a control vector. That is, in this embodiment, the administration of the viral vector decreases levels of PCNA by about 4 to about 12-fold, including values and ranges thereof, in the neuronal cells compared to levels of PCNA prior to introduction of the viral vector or compared to levels of PCNA in cells transduced with a control vector. In some embodiments, the administration of the viral vector decreases levels of PCNA by about 4 to about 12-fold, about 4 to about 11.5-fold, about 4 to about 11-fold, about 4 to about 10.5-fold, about 4 to about 10-fold, about 4 to about 9.5-fold, about 4 to about 9-fold, about 4 to about 8.5-fold, about 4 to about 8-fold, about 4 to about 7.5-fold, about 4 to about 7-fold, about 4 to about 6.5-fold, about 4 to about 6-fold, about 4 to about 5.5-fold, about 4 to about 5-fold, about 5 to about 12-fold, about 5 to about 11.5-fold, about 5 to about 11-fold, about 5 to about 10.5-fold, about 5 to about 10-fold, about 5 to about 9.5-fold, about 5 to about 9-fold, about 5 to about 8.5-fold, about 5 to about 8-fold, about 5 to about 7.5-fold, about 5 to about 7-fold, about 5 to about 6.5-fold, about 5 to about 6-fold, about 5 to about 5.5-fold, about 6 to about 12, about 6 to about 11.5-fold, about 6 to about 11-fold, about 6 to about 10.5-fold, about 6 to about 10-fold, about 6 to about 9.5-fold, about 6 to about 9-fold, about 6 to about 8.5-fold, about 6 to about 8-fold, about 6 to about 7.5-fold, about 6 to about 7-fold, about 6 to about 6.5-fold, about 7 to 12-fold, about 7 to about 11.5-fold, about 7 to about 11-fold, about 7 to about 10.5-fold, about 7 to about 10-fold, about 7 to about 9.5-fold, about 7 to about 9-fold, about 7 to about 8.5-fold, about 7 to about 8-fold, about 7 to about 7.5-fold, about 8 to about 12-fold, about 8 to about 11.5-fold, about 8 to about 11-fold, about 8 to about 10.5-fold, about 8 to about 10-fold, about 8 to about 9.5-fold, about 8 to about 9-fold, about 8 to about 8.5-fold, about 9 to about 12-fold, about 9 to about 11.5-fold, about 9 to about 11-fold, about 9 to about 10.5-fold, about 9 to about 10-fold, about 9 to about 9.5-fold, about 10 to 12-fold, about 10 to about 11.5-fold, about 10 to about 11-fold, about 10 to about 10.5-fold, about 11 to about 12-fold, about 11 to about 11.5-fold, including values and ranges thereof, compared to levels of PCNA prior to introduction of the viral vector or compared to levels of PCNA in cells transduced with a control vector. In some embodiments, the introduction of the viral vector decreases levels of PCNA by about 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 10.5-fold, 11-fold, 11.5-fold or by about 12-fold, compared to levels of PCNA prior to introduction of the viral vector or compared to levels of PCNA in cells transduced with a control vector.

In some embodiments, the introduction of the viral vector into the neuronal cell inhibits CRNA in the cell by about 30-80%, including values and ranges thereof, as measured by a decrease in levels of PCNA compared to levels of PCNA prior to introduction of the viral vector or compared to levels of PCNA in cells transduced with a control vector. That is, in this embodiment, the introduction of the viral vector decreases levels of PCNA by about 30-80% %, including values and ranges thereof, in the neuronal cells compared to levels of PCNA prior to introduction of the viral vector or compared to levels of PCNA in cells transduced with a control vector. In some embodiments, the introduction of the viral vector decreases levels of PCNA by about 30-80%, 30-75%, 30-70%, 30-65%, 30-60%, 30-55%, 30-50%, 30-45%, 35-80%, 35-75%, 35-70%, 35-65%, 35-60%, 35-55%, 35-50%, 40-80%, 40-75%, 40-70%, 40-65%, 40-60%, 40-50%, 50-80%, 50-75%, 50-70%, 50-65%, 50-60%, 60-80%, 60-75%, 60-70%, 65-80%, or 70-80%, including values and ranges thereof, compared to levels of PCNA prior to introduction of the viral vector or compared to levels of PCNA in cells transduced with a control vector. In some embodiments, the introduction of the viral vector decreases levels of PCNA by about 30%, 35%, 40%, 45%, 50%, 55%, 60%, 65%, 70%, 75%, or by about 80%, including values and ranges thereof, compared to levels of PCNA prior to introduction of the viral vector or compared to levels of PCNA in cells transduced with a control vector.

In some embodiments, the introduction of the viral vector into the neuronal cell inhibits CRNA in the neuronal cell by about 3 to about 15-fold, including values and ranges thereof, as measured by a decrease in levels of cleaved caspase-3 when compared to levels of cleaved caspase-3 prior to introduction of the viral vector or compared to levels of cleaved caspase-3 in cells transduced with a control vector. That is, in this embodiment, the introduction of the viral vector into the neuronal cell decreases levels of cleaved caspase-3 by about 3 to about 15-fold, including values and ranges thereof, in the neuronal cells compared to levels of cleaved caspase-3 prior to introduction of the viral vector or compared to levels of cleaved caspase-3 in cells transduced with a control vector. In some embodiments, the introduction of viral vector decreases levels of cleaved caspase-3 by about 3 to about 15-fold, about 3 to about 14.5-fold, about 3 to about 14-fold, about 3 to about 13.5-fold, about 3 to about 13-fold, about 3 to about 12.5-fold, about 3 to about 12-fold, about 3 to about 11.5-fold, about 3 to about 11-fold, about 3 to about 10.5-fold, about 3 to about 10-fold, about 3 to about 9.5-fold, about 3 to about 9-fold, about 3 to about 8.5-fold, about 3 to about 8-fold, about 3 to about 7.5-fold, about 3 to about 7-fold, about 3 to about 6.5-fold, about 3 to about 6-fold, about 3 to about 5.5-fold, about 3 to about 5-fold, about 3 to about 4.5-fold, about 3 to about 4-fold, about 4 to about 15-fold, about 4 to about 14.5-fold, about 4 to about 14-fold, about 4 to about 13.5-fold, about 4 to about 13-fold, about 4 to about 12.5-fold, 4 to about 12-fold, about 4 to about 11.5-fold, about 4 to about 11-fold, about 4 to about 10.5-fold, about 4 to about 10-fold, about 4 to about 9.5-fold, about 4 to about 9-fold, about 4 to about 8.5-fold, about 4 to about 8-fold, about 4 to about 7.5-fold, about 4 to about 7-fold, about 4 to about 6.5-fold, about 4 to about 6-fold, about 4 to about 5.5-fold, about 4 to about 5-fold, about 5 to about 15-fold, about 5 to about 14.5-fold, about 5 to about 14-fold, about 5 to about 13.5-fold, about 5 to about 13-fold, about 5 to about 12.5-fold, about 5 to about 12-fold, about 5 to about 11.5-fold, about 5 to about 11-fold, about 5 to about 10.5-fold, about 5 to about 10-fold, about 5 to about 9.5-fold, about 5 to about 9-fold, about 5 to about 8.5-fold, about 5 to about 8-fold, about 5 to about 7.5-fold, about 5 to about 7-fold, about 5 to about 6.5-fold, about 5 to about 6-fold, about 5 to about 5.5-fold, about 6 to about 15-fold, about 6 to about 14.5-fold, about 6 to about 14-fold, about 6 to about 13.5-fold, about 6 to about 13-fold, about 6 to about 12.5-fold, about 6 to about 12, about 6 to about 11.5-fold, about 6 to about 11-fold, about 6 to about 10.5-fold, about 6 to about 10-fold, about 6 to about 9.5-fold, about 6 to about 9-fold, about 6 to about 8.5-fold, about 6 to about 8-fold, about 6 to about 7.5-fold, about 6 to about 7-fold, about 6 to about 6.5-fold, about 7 to about 15-fold, about 7 to about 14.5-fold, about 7 to about 14-fold, about 7 to about 13.5-fold, about 7 to about 13-fold, about 7 to about 12.5-fold, about 7 to 12-fold, about 7 to about 11.5-fold, about 7 to about 11-fold, about 7 to about 10.5-fold, about 7 to about 10-fold, about 7 to about 9.5-fold, about 7 to about 9-fold, about 7 to about 8.5-fold, about 7 to about 8-fold, about 7 to about 7.5-fold, about 8 to about 15-fold, about 8 to about 14.5-fold, about 8 to about 14-fold, about 8 to about 13.5-fold, about 8 to about 13-fold, about 8 to about 12.5-fold, about 8 to about 12-fold, about 8 to about 11.5-fold, about 8 to about 11-fold, about 8 to about 10.5-fold, about 8 to about 10-fold, about 8 to about 9.5-fold, about 8 to about 9-fold, about 8 to about 8.5-fold, about 9 to about 15-fold, about 9 to about 14.5-fold, about 9 to about 14-fold, about 9 to about 13.5-fold, about 9 to about 13-fold, about 9 to about 12.5-fold, about 9 to about 12-fold, about 9 to about 11.5-fold, about 9 to about 11-fold, about 9 to about 10.5-fold, about 9 to about 10-fold, about 9 to about 9.5-fold, about 10 to about 15-fold, about 10 to about 14.5-fold, about 10 to about 14-fold, about 10 to about 13.5-fold, about 10 to about 13-fold, about 10 to about 12.5-fold, about 10 to 12-fold, about 10 to about 11.5-fold, about 10 to about 11-fold, about 10 to about 10.5-fold, about 11 to about 15-fold, about 11 to about 14.5-fold, about 11 to about 14-fold, about 11 to about 13.5-fold, about 11 to about 13-fold, about 11 to about 12.5-fold, about 11 to about 12-fold, about 11 to about 11.5-fold, about 12 to about 15-fold, about 12 to about 14.5-fold, about 12 to about 14-fold, about 12 to about 13.5-fold, about 12 to about 13-fold, about 12 to about 12.5-fold, about 13 to about 15-fold, about 13 to about 14.5-fold, about 13 to about 14-fold, about 13 to about 13.5-fold, about 14 to about 15-fold, about 14 to about 14.5-fold, including values and ranges thereof, compared to levels of cleaved caspase-3 prior to introduction of the viral vector or compared to levels of cleaved caspase-3 in cells transduced with a control vector. In some embodiments, the introduction of the viral vector decreases levels of cleaved caspase-3 by about 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 10.5-fold, 11-fold, 11.5-fold, 12-fold, 12.5-fold, 13-fold, 13.5-fold, 14-fold, 14.5-fold or by about 15-fold compared to levels of cleaved caspase-3 prior to introduction of the viral vector or compared to levels of cleaved caspase-3 in cells transduced with a control vector.

In some embodiments, the introduction of the viral vector into the neuronal cell inhibits CRNA in the patient by about 20-90%, including values and ranges thereof, as measured by a decrease in levels of cleaved caspase-3 when compared to levels of cleaved caspase-3 prior to introduction of the viral vector or compared to levels of cleaved caspase-3 in cells transduced with a control vector. That is, in this embodiment, the introduction of the viral vector into the neuronal cell decreases levels of cleaved caspase-3 by about 20-90%, including values and ranges thereof, in the cell, compared to levels of cleaved caspase-3 prior to introduction of the viral vector or compared to levels of cleaved caspase-3 in cells transduced with a control vector. In some embodiments, the introduction of the viral vector decreases levels of cleaved caspase-3 by about 20-90%, about 20-90%, 20-85%, 20-80%, 20-75%, 20-70%, 20-65%, 20-60%, 20-55%, 20-50%, 20-45%, 20-40%, 20-30%, 25-90%, 25-85%, 25-80%, 25-75%, 25-70%, 25-65%, 25-60%, 25-55%, 25-50%, 25-45%, 25-40%, 25-35%, 30-90%, 30-85%, 30-80%, 30-75%, 30-70%, 30-65%, 30-60%, 30-55%, 30-50%, 30-45%, 35-90%, 35-85%, 35-80%, 35-75%, 35-70%, 35-65%, 35-60%, 35-55%, 35-50%, 40-90%, 40-85%, 40-80%, 40-75%, 40-70%, 40-65%, 40-60%, 40-50%, 50-90%, 50-95%, 50-80%, 50-75%, 50-70%, 50-65%, 50-60%, 60-90%, 60-85%, 60-80%, 60-75%, 60-70%, 65-90%, 65-85%, 65-80%, 70-90%, 70-85%, 70-80%, 80-90%, including values and ranges thereof, compared to levels of cleaved caspase-3 prior to introduction of the viral vector or compared to levels of cleaved caspase-3 in cells transduced with a control vector. In some embodiments, the introduction of the viral vector decreases levels of cleaved caspase-3 by about 20%, 25%, 30%, 35%, 40%, 45%, 50%, 55%, 60%, 65%, 70%, 75%, 80%, 85%, or by about 90%, including values and ranges thereof, compared to levels of cleaved caspase-3 prior to introduction of the viral vector or compared to levels of cleaved caspase-3 in cells transduced with a control vector.

The administration of the viral vector expressing miR-449a as described herein to a subject reduces the rate of neurodegeneration in the subject as measured by a decrease in the levels of PCNA or cleaved caspase-3 in the neuronal cells of the subject or by a decrease in the levels of cell cycle proteins such as CDC25A and cyclin D1 proteins in the neuronal cells of the subject. Accordingly, provided herein is a method of reducing neurodegeneration in a patient suffering from cognitive impairment or at a risk of developing AD, comprising administering to the patient a viral vector comprising a nucleotide sequence containing a miRNA-449a sequence described herein. The rate or the extent of neurodegeneration can be measured by measuring the levels of CDC25A protein, cyclin D1 protein, PCNA, cleaved and caspase-3 in a sample obtained from the subject.

In some embodiments, the administration of the viral vector comprising a nucleotide sequence containing a miRNA-449a sequence improves the cognitive function, learning abilities, and/or memory of the patients. In some embodiments, the administration of the viral vector improves a long-term and short-term memory in the patients.

In some embodiments of the present invention provides a composition comprising a viral vector comprising a nucleotide sequence containing a miR-449a sequence for use in treating a cognitive impairment due to miR-449a dysregulation.

In some embodiments, the miR-449a sequence comprises a miRNA-449 hairpin loop sequence including mature miR-449a sequence.

In some embodiments, the administration of the composition comprising a viral vector comprising a nucleotide sequence containing a miR-449a sequence increases levels of miR-449a in neuronal cells of the patient.

In some embodiments, the viral vector of the composition is a lentiviral vector.

In some embodiments, the composition is in an injection or an infusion form.

In some embodiments, the composition decreases the levels of CDC25A proteins in neuronal cells of a patient by about 2 to about 6-fold, including sub-ranges and values described herein, compared to levels of CDC25A proteins prior to administration of the composition.

In some embodiments, the composition decreases the levels of CDC25A proteins in neuronal cells of a patient by about 30-80%, including sub-ranges and values described herein, compared to levels of CDC25A proteins prior to administration of the composition.

In some embodiments, the composition decreases the levels of cyclin D1 proteins in neuronal cells of a patient by about 2 to about 8-fold, including sub-ranges and values described herein, compared to levels of cyclin D1 proteins prior to administration of the composition.

In some embodiments, the composition decreases the levels of cyclin D1 proteins in neuronal cells of a patient by about 20-90%, including sub-ranges and values described herein, compared to levels of cyclin D1 proteins prior to administration of the composition.

In some embodiments, the composition inhibits cell cycle related neuronal apoptosis (CRNA) in a patient by about 4 to 12-fold, including sub-ranges and values described herein, as measured by a decrease in levels of proliferating cell nuclear antigen (PCNA) when compared to levels of PCNA prior to administration of the composition.

In some embodiments, the composition inhibits cell cycle related neuronal apoptosis (CRNA) in the patient by about 30-80%, including sub-ranges and values described herein, as measured by a decrease in levels of proliferating cell nuclear antigen (PCNA) when compared to levels of PCNA prior to administration of the composition.

In some embodiments, the composition inhibits cell cycle related neuronal apoptosis (CRNA) in a patient by about 3 to 15-fold, including sub-ranges and values described herein, as measured by a decrease in levels of cleaved caspase 3 when compared to levels of cleaved caspase 3 prior to administration of the composition.

In some embodiments, the composition inhibits cell cycle related neuronal apoptosis (CRNA) in the patient by about 20-90%, including sub-ranges and values described herein, as measured by a decrease in levels of cleaved caspase 3 when compared to levels of cleaved caspase 3 prior to administration of the composition.

In some embodiments, the composition improves short-term and long-term memory in a patient.

In some embodiments, the composition improves the learning ability of the patient.

In some embodiments, the composition is used for the treatment of the cognitive impairment due to miR-449a dysregulation associated with a degenerative neurological disorder.

In some embodiments, the composition is used for the treatment of the degenerative neurological disorder such as Alzheimer's disease.

Alzheimer's disease has a spectrum of clinical stages depending on the severity of cognitive and/or functional impairment. Some of the clinical stages of AD include, but are not limited to, a pre-clinical stage, mild cognitive impairment (MCI), mild AD, moderate AD, and an advanced AD. In some embodiments, the present disclosure provides a method for treating a subject having a pre-clinical stage of AD, comprising administering to the subject a viral vector comprising a nucleotide sequence containing a miRNA-449a sequence described herein. In some embodiments, the present disclosure provides a method for treating a subject having a mild cognitive impairment (MCI), comprising administering to the subject said viral vector.

In some embodiments, the present disclosure provides use of a viral vector comprising a miRNA-449a sequence described herein for treating cognitive impairment. AD is a progressive disease related to the nervous system and can be categorized into various stages based on the severity of symptoms as described herein. The present disclosure contemplates use of a viral vector comprising a nucleotide sequence containing a mature miRNA-449a sequence for treating various stages of AD described herein. Dosage forms, and routes of administration of the viral vector are discussed above.

In some embodiments, the present disclosure provides a viral vector comprising a nucleotide sequence containing a miRNA-449a sequence for use as a medicament for treating various stages of cognitive impairment due to miR-449a dysregulation. Dosage forms and routes of administration that may be employed for the medicament are discussed above.

Methods for preparing lentiviral vectors for expressing miRNAs are known in the art. In an exemplary embodiment, a lentivirus system comprising oligonucleotides containing miRNA hairpin loop sequence (obtained from a miRNA database) with EcoRI and PacI restriction site overhangs is synthesized as follows: Oligos are annealed and cloned in a vector such as pLKO.3G vector and this plasmid construct along with packaging plasmids pCMV-vsv-g and pAR8.2-dvpr are co-transfected in HEK293T cells (Stewart, Dykxhoorn et al. 2003, Tiscornia, Singer et al. 2006). Supernatant from the cells is collected 40 and 72 h post transfection, filtered and are used to estimate the number of transducing units (TU/ml) after 24-48 h. Based on the titre of transducing units, a desired amount of the supernatant is used to transduce neuronal cells in vitro or is administered to the subject. If required, the supernatant can be concentrated using a concentrator. In an exemplary embodiment, a concentration of ˜10⁶ to 10⁷ TU/ml is employed for administering to the subjects.

It is to be understood that the foregoing descriptive matter is illustrative of the disclosure and not a limitation. While considerable emphasis has been placed herein on the particular features of this disclosure, it will be appreciated that various modifications can be made, and that many changes can be made in the preferred embodiments without departing from the principles of the disclosure. Those skilled in the art will recognize that the embodiments herein can be practiced with modification within the spirit and scope of the embodiments as described herein. Similarly, additional embodiments and features of the present disclosure will be apparent to one of ordinary skill in art based upon description provided herein.

Descriptions of well-known/conventional methods/steps and techniques are omitted so as to not unnecessarily obscure the embodiments herein. Further, the disclosure herein provides for examples illustrating the above-described embodiments, and in order to illustrate the embodiments of the present disclosure certain aspects have been employed. The examples used herein for such illustration are intended merely to facilitate an understanding of ways in which the embodiments herein may be practiced and to further enable those of skill in the art to practice the embodiments herein. Accordingly, the following examples should not be construed as limiting the scope of the embodiments herein.

### EXAMPLES

### Example 1

### Methods and Materials

### (i) APP/PS1 (TgAD) Alzheimer's Disease Mouse Model

Amyloid Precursor Protein/Presenillin 1 (PS1) transgenic (TgAD) mouse model for AD (strain B6C3-Tg APPswe, PSEN1dE9 85 Dbo/J; stock number 004462) maintained at the Jackson laboratory was a kind gift from National Brain Research Centre, Manesar to NII, New Delhi. The TgAD mice were double transgenic for amyloid-β precursor protein/presenilin 1 (PS1). They express a chimeric mouse/human amyloid-β precursor protein containing the K595N and M596L; two Swedish mutations and a mutant human presenilin1 gene carrying the exon-9 deletion under the control of mouse prion promoter elements, directing transgene expression predominantly to the central nervous system neurons. The levels of Aβ42 produced are significantly higher in female animals (Jankowsky, Slunt et al. 2001, Jankowsky, Fadale et al. 2004). Wild type and transgenic (TgAD) mice were genotyped using genomic DNA isolated from mouse tail and PCR primers according to the Jackson's laboratory protocol.

### (ii) Cell Culture

Cortical neurons from Embryonic day 18 (E18) Sprague-Dawley rats or Embryonic day 16 (E16) APP/PS1 transgenic AD mice were isolated and cultured as previously published (Modi, Komaravelli et al. 2012, Modi, Jaiswal et al. 2016). Briefly, E18 rat or E16 mouse embryos were dissected, and cortical region of the brain was isolated and treated with Trypsin-DNase followed by addition of Serum Containing Media (SCM) and centrifugation at 500×g for 5 min at room temperature. Cell pellet was resuspended in SCM and plated on poly-L-Lysine coated 6-well plates. After 12h, cells were washed with Tyrode's CMF PBS supplemented with glucose and NaHCO3 and were maintained in Serum Free Medium (SFM) containing B27 and N2 supplement (Gibco, Life technologies), 1× penicillin-streptomycin, L-glutamine, and glucose in a CO₂ incubator maintaining 5% CO₂ levels, for five days. Typically, in vitro transfections or Aβ₁₋₄₂ treatments were performed at DIV5.

HEK293T/A (Human Embryonic Kidney) cells were maintained in DMEM with 10% fetal bovine serum and 1× antibiotic/antimycotic at 37° C. in 5% CO₂.

All experiments were performed in accordance with the guidelines for animal experiments of the Institutional Animal Ethical Committee of the National Institute of Immunology.

(iii) RNA Isolation and qRT-PCR

After the desired treatments, total RNA was isolated by using TRIzol reagent (Thermo Fisher Scientific, USA). Briefly, the samples in TRIzol reagent were homogenized and kept at room temperature for 5 min, followed by a chloroform wash. 3M sodium acetate (pH 5.2) and 1 μl of 50 mg/ml glycogen (for small RNA isolation only) and isopropanol were added for precipitating total RNA which was washed twice with 75% ethanol, followed by resuspension in nuclease free water.

Quantitative RT-PCR (qRT-PCR) for microRNA was performed by using a TaqMan microRNA assay kit. Typically, 1 μg of total RNA was reverse transcribed using a TaqMan microRNA RT kit (Applied Biosystems) and microRNA-specific stem-loop RT primers provided in the TaqMan microRNA assay kit. Briefly, following the RT step, 1.33 μl of the RT reaction product was combined with 1 μl of a TaqMan microRNA assay (20×; forward primer, reverse primer, and probe, Applied Biosystems, catalog no. 4427975, hsa-miR-449a assay #001030 or hsa-miR-16-5p assay #000391 or RNU6B assay #001093) and 10 μl of TaqMan universal PCR master mix, No AmpErase UNG, in a 20 μl final volume. Realtime PCR was performed in CFX96 real time thermo cycler (BioRad). The expression of miRNA was defined on the basis of threshold cycle (C_{T}), and the relative expression levels were determined by ΔΔC_{T} after normalization with RNU6B.

### (iv) Transfection and Treatments

Lipofectamine 2000 reagent (Invitrogen) was used for transfection of plasmid DNA and siRNA according to manufacturer's instructions. Cortical neurons were transfected with 1-3 μg of plasmid DNA or 100 pmoles of anti-miRNA per well in a six well plate in serum-free medium without antibiotic. After 3-4 hours of transfection, cultures were moved to medium with supplements and antibiotic. Typically, 0.5 μM of soluble oligomers of Aβ₁₋₄₂ (R-peptide) was used for the treatment of cortical neurons for 48 h, to induce CRNA as described previously (Stine, Dahlgren et al. 2003, Modi, Komaravelli et al. 2012, Modi, Jaiswal et al. 2016).

### (v) Viral Production and Transductions

Two types of viral gene delivery systems were used in this study. Lentivirus system was used for miRNA expression (Vilardo, Barbato et al. 2010) and adenovirus system for target gene expressions (Modi, Komaravelli et al. 2012) in post-mitotic neurons.

Lentivirus: Oligonucleotides containing miRNA hairpin loop sequence (obtained from miRDB) or unrelated sequence (negative control) sequence with EcoRI and PacI restriction site overhangs were commercially synthesized from Merck (NJ, USA). Oligos were annealed and cloned in pLKO.3G vector and this plasmid construct along with packaging plasmids pCMV-vsv-g and pAR8.2-dvpr were co-transfected in HEK293T cells (Stewart, Dykxhoorn et al. 2003, Tiscornia, Singer et al. 2006). Supernatant from the cells was collected 40 and 72 h post transfection, filtered and used to estimate the number of transducing units (TU/ml) by GFP fluorescence after 24-48 h. This supernatant was further used to transduce cultured primary cortical neurons. Lenti-X concentrator solution (Takara) was used for concentrating the virus to be injected in the brains at a concentration of ˜10⁶ to 10⁷ TU/ml.

Adenovirus: Adenovirus constructs were prepared for CDC25A and cyclin D1 (details in Supp, methods) expression in post mitotic neurons. pAd Track shuttle vector constructs were generated by cloning relevant cDNA and the resultant plasmids were digested with Pmel followed by electroporation in Escherichia coli BJ5138 containing pAdEasy-1 vector. The recombinant clones were digested with Pac I and transfected in HEK293A cells. The adenovirus was harvested and amplified using standard procedures (He, Zhou et al. 1998, Luo, Deng et al. 2007, Lock, Alvira et al. 2010). Adenovirus, at ˜10 multiplicity of infection (MOI), was incubated with cortical neurons and efficiency of infection was determined by observing GFP fluorescence after 24-48 h.

### (vi) Immunoblotting

Cells were washed with 1×PBS and lysed using ice cold lysis buffer (100 mM Tris-HCl PH 7.4, 5 mM EDTA, 100 mM NaCl, 1% Triton×100 and 10% Glycerol, 1 mM phenyl methane sulfonyl fluoride (PMSF), 1 mM sodium orthovanadate, 20 mM β-glycero-phosphate and 1× protease inhibitor cocktail was added before use). Immunoblotting was performed as described previously (Modi, Jaiswal et al. 2016) using primary antibodies and secondary antibody conjugated with horse radish peroxidase (HRP). Chemiluminescence reagent West Pico or West Dura (Pierce) was used for detection as per manufacturer's instructions.

(vii) BrdU Incorporation and TUNEL Assay

5-bromo-2′-deoxyuridine (BrdU) labelling was performed to detect DNA replication. Anti-BrdU antibody (GE) was used to detect incorporated BrdU (Modi, Komaravelli et al. 2012, Modi, Jaiswal et al. 2016). Terminal deoxynucleotidyl transferase dUTP nick end labelling (TUNEL) assay to detect cell death was performed by using Dead End fluorometric TUNEL system (G3250, Promega) as per manufacturer's guidelines and Hoechst 33342 (Molecular Probes) was used to stain the nuclei. These two assays were performed simultaneously (Modi, Jaiswal et al. 2016, Chauhan, Modi et al. 2020) and labelled cells were visualized using a Zeiss Axiolmager1 microscope and Axiovision software was used for image acquisition and processing images and population of cells positive for BrdU and/or TUNEL was determined.

(viii) Luciferase Reporter Assays

Cyclin D1/CDC25A 3′-UTR was amplified using primers mentioned in Supplementary Table S3 using rat cDNA template and cloned downstream of the Renilla luciferase gene in psiCHECK2 plasmid containing synthetic firefly luciferase gene (transfection control). The miRNA binding sites in 3′-UTR was mutated for each UTR construct (see details in supplementary methods). Transfections of these plasmid constructs in rat cortical neurons was performed using Lipofectamine 2000 and cells were harvested after 48 h and frozen at −80° C. for at least 24 h. After brief centrifugation, luciferase and renilla activity were measured with a dual-luciferase reporter assay kit (Promega, Madison, WI).

### (ix) Lentivirus Stereotaxic Injections

WT/TgAD6 month old female mice were anaesthetized with a ketamine/xylazine solution (75 mg/kg ketamine+10 mg/kg xylazine, intramuscular injection). Lentivirus encoding miR-449a precursor sequence or a negative control sequence containing ˜10⁶-10⁷ TU/ml, in a final volume of 3 μl, were stereotaxically injected in both hemispheres of the cortex at the following coordinates as anterior/posterior: +1.8 mm; mediolateral: +/−1.9 mm; dorsal/ventral: −1.2 mm to the Bregma (Saunders, Oldenburg et al. 2015). The viral suspensions were injected at the flow rate 1 μl/min (Stoelting, USA), the needle-wound was closed and skin was sutured. Animals were monitored during recovery and subject to behavioral tests after 45 days.

### Results

### Example-2: MiR-449a Regulates Neuronal Differentiation by Suppressing Neuronal Cell Cycle

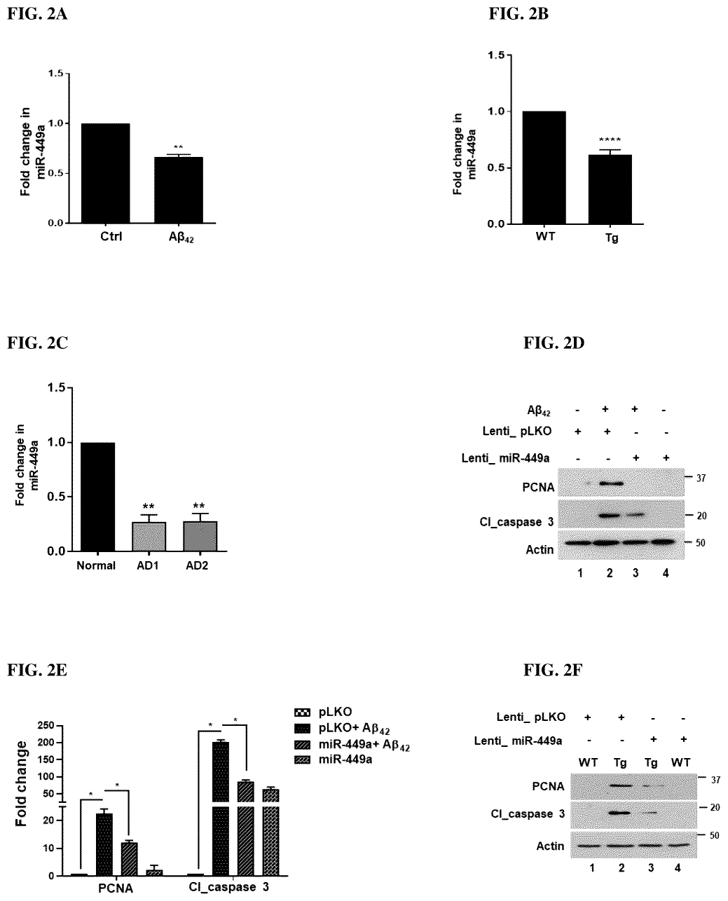

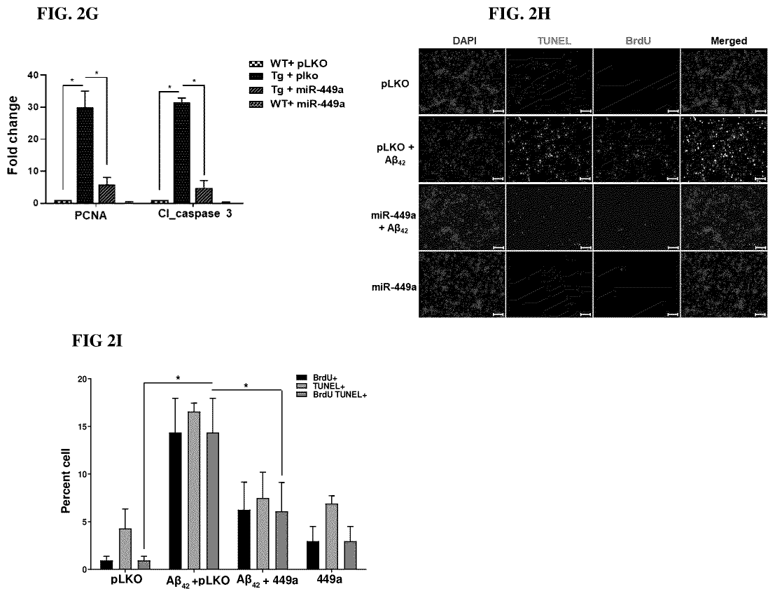

One of the major objectives of the present study was to decipher a connection between cell cycle and death of neurons. Therefore, we first investigated if miR-449a regulate the cell cycle during neuronal differentiation. The expression of these miRNA was determined during differentiation of neurons. For this purpose, primary cortical neurons were allowed to differentiate for 7 days in vitro, qRT-PCR revealed that the levels of miR-449a significantly increased with differentiation (FIG. 2A).

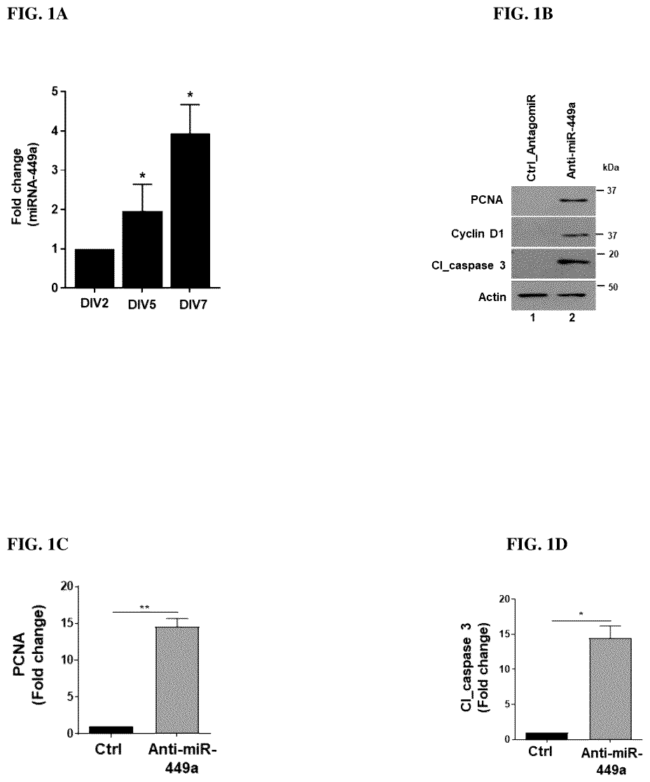

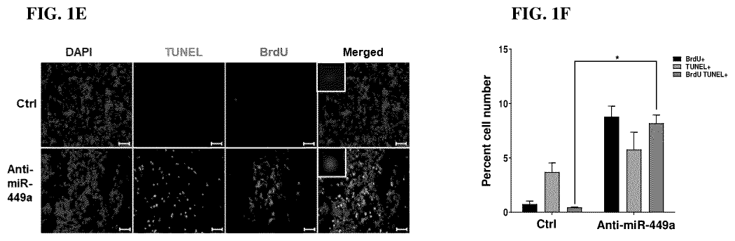

In the light of this observation, it was pertinent to test if this miRNA regulate the cell cycle during neuronal differentiation, which was done by assessing the levels of cyclin D1 (early G1 marker) and PCNA (S-phase marker) that decrease upon differentiation of neurons as they exit the cell cycle (Modi, Jaiswal et al. 2016). Rat cortical neurons were transfected with antagomir (anti-miR) to inhibit the expression of miR-449a followed by Western blotting, which revealed that cyclin D1 and PCNA that were expressed at very low levels or were almost absent in differentiated neurons (Modi, Komaravelli et al. 2012, Modi, Jaiswal et al. 2016) (FIG. 1B lane 1), were elevated upon miR-449a inhibition (FIG. 1B lane 2). It was also interesting to note that the apoptotic marker, cleaved caspase 3 was simultaneously upregulated upon miR-449a inhibition. To further confirm these observations, BrdU labeling and TUNEL assay were performed, which are indicative of DNA replication and apoptosis, respectively. A significant increase in the fraction of cells which were BrdU⁺ as well as TUNEL⁺ was observed upon miR-449a inhibition (FIG. 1E), which suggested that there was an increase in reactivation of cell cycle and apoptosis in these cells. Collectively, these data indicated that miR-449a keeps the neuronal cell cycle suppressed, which is critical for maintaining the state of differentiation failing which neurons undergo apoptosis.

### Example 3: MiR-449a is Deregulated in Response to Aβ42

qRT-PCR was performed to determine the expression of miR-449a in TgAD neurons as well as in rat cortical neurons treated with neurotoxic Aβ₄₂ peptide for 48 hours, which is known to cause CRNA (Varvel, Bhaskar et al. 2008, Modi, Komaravelli et al. 2012, Modi, Jaiswal et al. 2016, Chauhan, Modi et al. 2020). The expression of miR-449a was found to be significantly reduced in rat cortical neurons in response to Aβ₄₂ treatment (FIG. 2A). The expression of miR-449a was significantly reduced in TgAD neurons in comparison to WT littermates (FIG. 2B). These studies suggested that miR-449a is deregulated in response to Aβ₄₂. A frontal cortex tissue was obtained from two AD patients and age-matched normal individuals from a brain bank to assess the expression of these miRNAs. Interestingly, the expression of miR-449a was significantly lower in the cortex of two AD patients in comparison to healthy individuals (FIG. 2C), which supported studies performed in cultured neurons and raised the possibility of involvement of these miRNAs in the disease.

### Example 4: Deregulation of miR-449a Levels Regulate CRNA

The miR-449a was deregulated in response to Aβ₄₂ (FIG. 2A-C), it was pertinent to check the outcome of its deregulation on cell cycle as these miRNAs suppress the neuronal cell cycle (FIG. 2) as well as neuronal survival. To address this, miRNA was over expressed using lentiviral transduction in rat cortical neurons followed by Aβ₄₂ treatment or in neurons from TgAD mice. Previous studies have indicated that neurons undergo CRNA in these situations (Modi, Jaiswal et al. 2016, Chauhan, Modi et al. 2020).

Western blotting revealed that PCNA, which was elevated due to Aβ₄₂ treatment, was significantly downregulated upon miR-449a (FIG. 2D, lane 3 vs. lane 2) along with reduced cleaved caspase 3 (FIG. 2D, lane 3 vs. lane 2). Similar observations were made when miR-449a was overexpressed using lentiviral transduction in TgAD mouse cortical neurons as PCNA and cleaved caspase 3 levels were reduced (FIG. 2G).

BrdU incorporation and TUNEL labelling was performed as described above to detect the status of cell cycle and apoptosis, respectively. Aβ₄₂ treatment resulted in a significant increase TUNEL⁺/BrdU⁺ neurons consistent with previous studies and indicated that these neurons exhibited aberrant cell cycle reentry which results in apoptosis (CRNA) (Modi, Komaravelli et al. 2012, Modi, Jaiswal et al. 2016, Chauhan, Modi et al. 2020). A significant reduction in BrdU⁺/TUNEL⁺ cells was observed in Aβ₄₂-treated cortical neurons in presence of miR-449a (FIG. 2I). Collectively, these findings established that miR-449a plays an important role in preventing aberrant cell cycle reentry of neurons which is induced by Aβ₄₂ and results in apoptosis.

### Example 5: MiR-449a May Prevent CRNA by Targeting Cyclin D1 and CDC25A

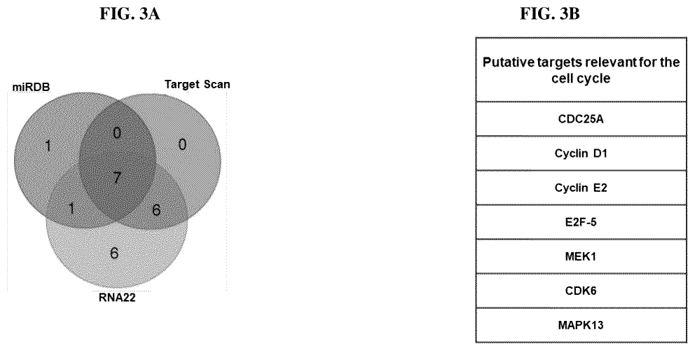

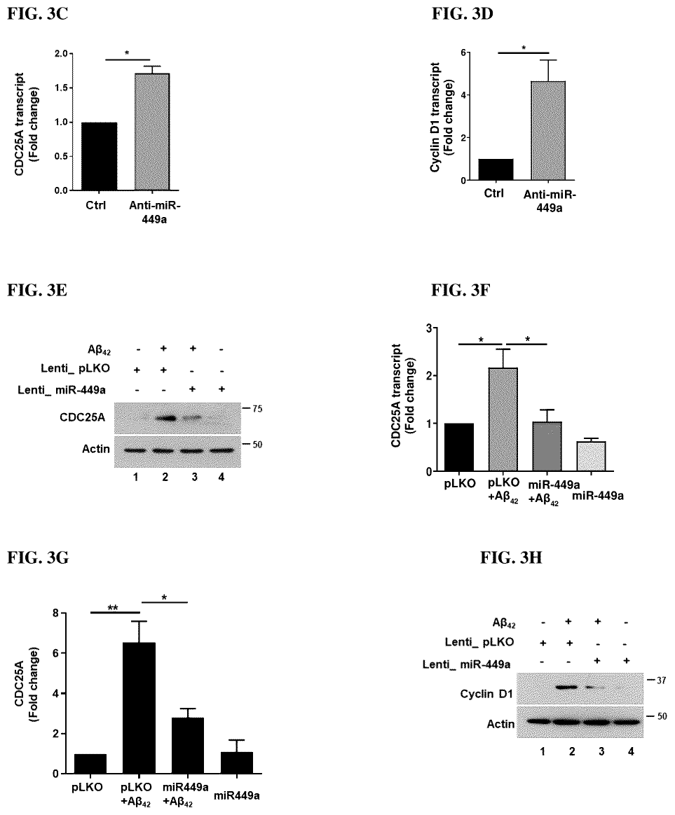

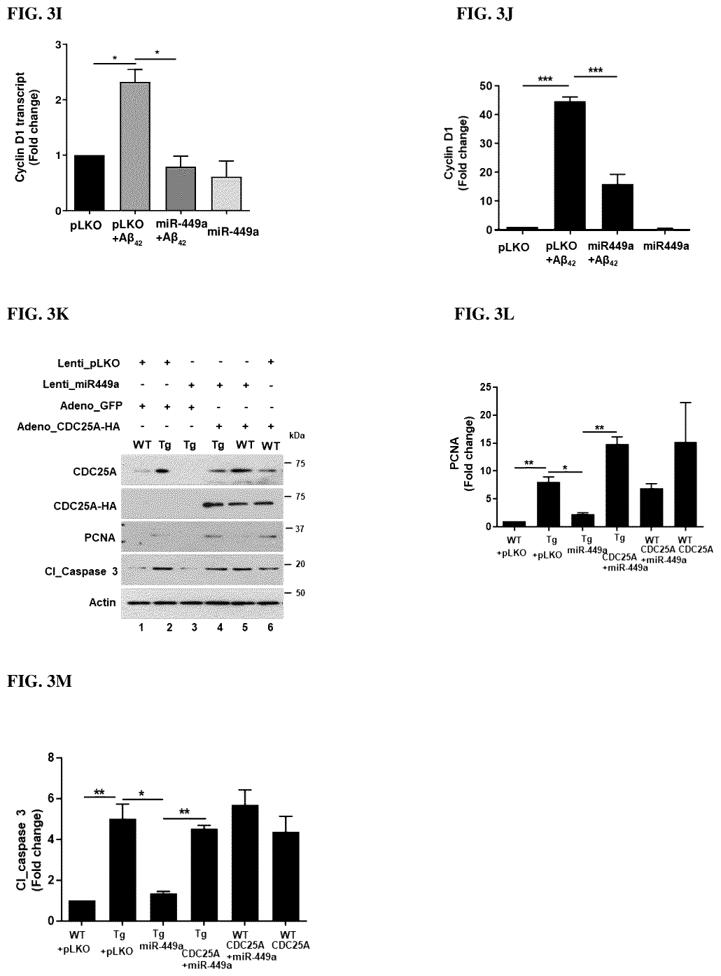

In order to decipher the mechanism via which miR-449a regulates the cell cycle during neuronal differentiation, the targets of this miRNA were predicted using in silico analysis. It was found that it has putative target sites in 3′-UTR of various genes that have been associated with cell cycle progression, which include cyclin D1, CDC25A, cyclin E2, CDK6 (FIGS. 3A and B). Cyclin D1 is involved in G1 progression as it activates CDK4/6, and CDC25A is a phosphatase involved in cell cycle regulation as it activates CDKs by removing phosphates from their inhibitory phosphorylation site (T14, Y15 in CDK1) (lavarone and Massague 1997, Biswas, Sanphui et al. 2017). Previous studies have indicated that miR-449a may target cyclin D1 and CDC25A in dividing cells (Ma, Li et al. 2011, Yuan, Shi et al. 2015, Li, Huang et al. 2016). Given that both cyclin D1 and CDC25A are critical for G1-S transition, it was reasonable to probe if miR-449a prevents CRNA by targeting these cell cycle regulators.

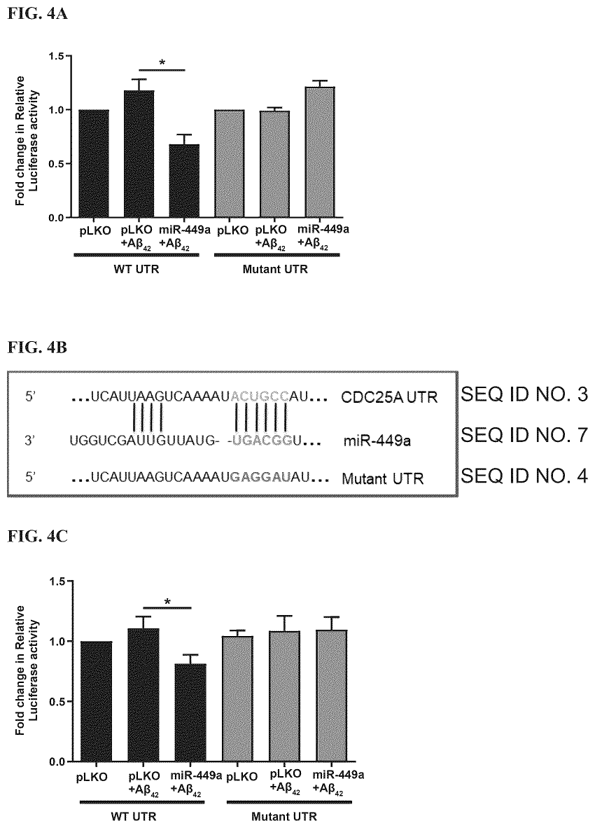

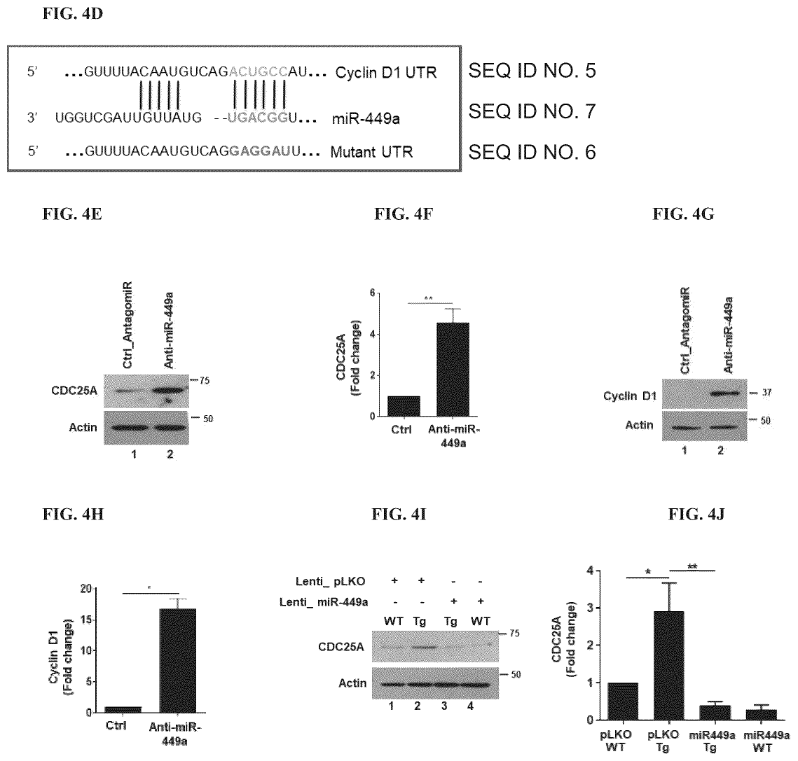

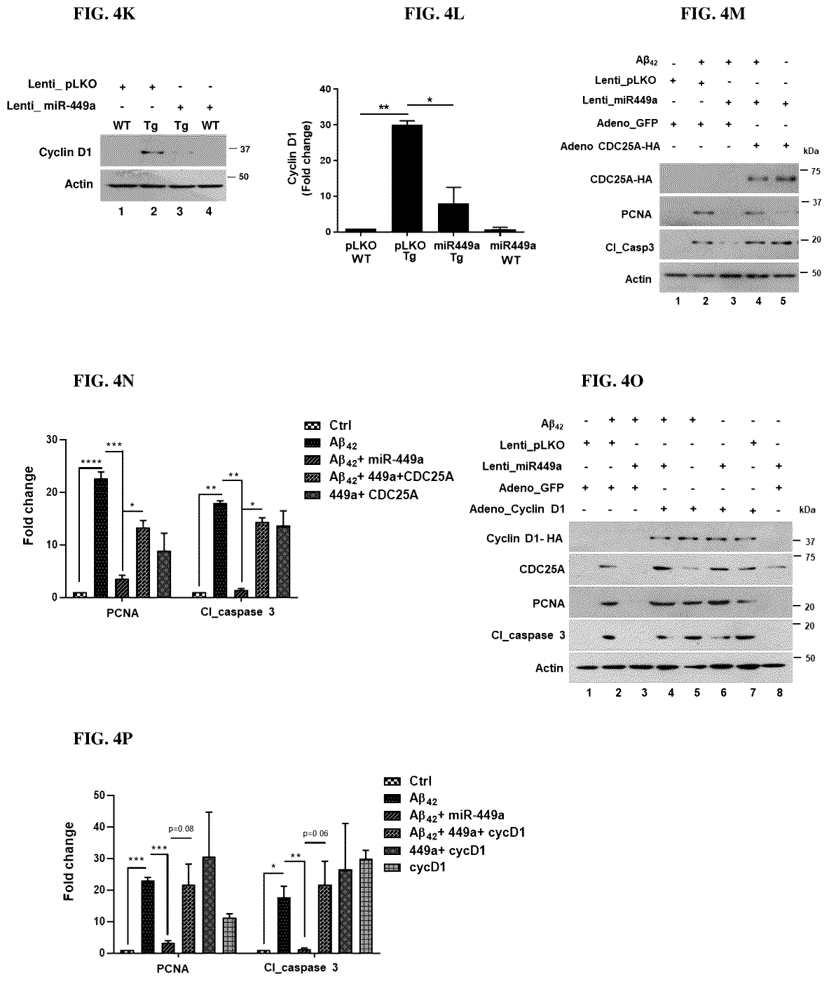

To study if miR-449a targets 3′ UTRs of cyclin D1 and CDC25A mRNAs, rat cortical neurons were transfected with luciferase reporter plasmid containing the 3′-UTR of cyclin D1 or CDC25A. The overexpression of miR-449a suppressed luciferase activity in both cases (FIG. 4A-D). In contrast, luciferase activity for the mutant 3′-UTR in which the miR-449a target site was disrupted in both CDC25A and cyclin D1, remained almost unchanged. Importantly, transfection of anti-miR-449a in cortical neurons caused a significant increase in cyclin D1 and CDC25A mRNA (FIG. 3C-D) as well as protein (FIG. 4E-G). Moreover, the overexpression of miR-449a caused a significant decrease in both cyclin D1 and CDC25A, which increased upon Aβ42 treatment (FIG. 3E-G) or was upregulated in TgAD neurons (FIG. 4I-L). These data suggested that miR-449a may repress the expression of key cell cycle regulators cyclin D1 and CDC25A in neurons and its downregulation by Aβ₄₂ may contribute to the increase in CDC25A and cyclin D1 expression.

Next, the ability of miR-449a to regulate CDC25A or cyclin D1 contributes to CRNA was tested. To this end, CDC25A was overexpressed using adenovirus in Aβ₄₂-treated (FIG. 4K) or TgAD (FIG. 3K) neurons. Since overexpressed CDC25A did not possess the 3′-UTR, its expression would not be influenced by miR-449a. The expression of PCNA and cleaved caspase 3 was determined, which increased in Aβ₄₂ treated neurons (FIG. 4K, lane 2) and miR-449a overexpression impaired the expression of these proteins (FIG. 4K, lane 3 v/s lane 2). Strikingly, when CDC25A was overexpressed along with miR-449a, the expression of these proteins was observed again (FIG. 4K, lane 4 v/s lane 3), which suggested that miR-449a mediated targeting of CDC25A may prevent CRNA. Similar experiments were performed to establish if miR-449a targeting of cyclin D1 also contributes to CRNA. In these experiments, while reversal of CRNA was observed in the case of neurons overexpressing both cyclin D1 and miR-449a in Aβ42-treated neurons (FIG. 4P lane 3 vs lane 4) but these effects (p=0.08) were marginally less significant in comparison to CDC25A (p<0.05) over expression (FIG. 4K). Collectively, these data indicated that the ability of miR-449a to repress at least CDC25A and possibly cyclin D1 significantly prevents aberrant cell cycle reentry and apoptosis of neurons by Aβ42.

### Example 6: Lentivirus Expressing miR-449a Improves Learning and Memory Defects in APP/PS1 (TgAD) Mice

Given that miR-449a expression is impaired in TgAD mouse neurons resulting in CRNA, it was pertinent to see if elevation in its expression prevents cognitive defects exhibited by these animals. Since previous studies (Gimbel, Nygaard et al. 2010) and our preliminary observations suggested that these defects are most apparent from ˜6 months of age, miR-449a was overexpressed in these animals of this age.

Stereotaxic injections were performed in the frontal cortex as: a. present studies have been performed on cortical neurons from TgAD mice and consistent with previous reports (Modi, Komaravelli et al. 2012, Modi, Jaiswal et al. 2016, Chauhan, Modi et al. 2020) these neurons exhibit CRNA; b. Previous studies on various AD mouse models have reported aberrant cell cycle re-entry of neurons in the cortex (Varvel, Bhaskar et al. 2008, Li, Cheung et al. 2011); c. The neuronal loss has been reported in the hippocampus as well as the cortex (Hyman, Phelps et al. 2012) and may collectively contribute to AD-related cognitive defects.

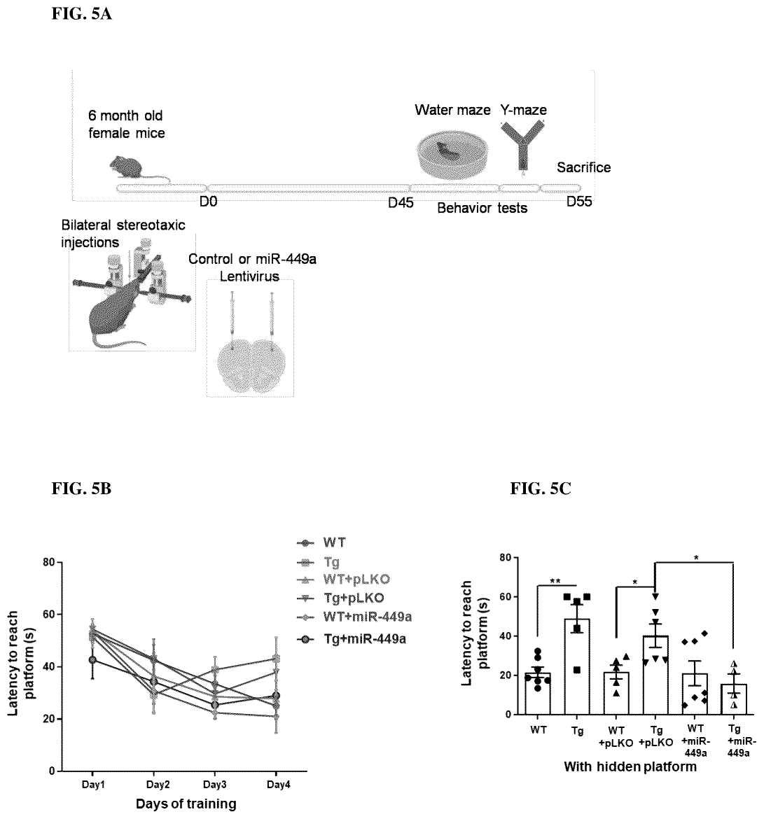

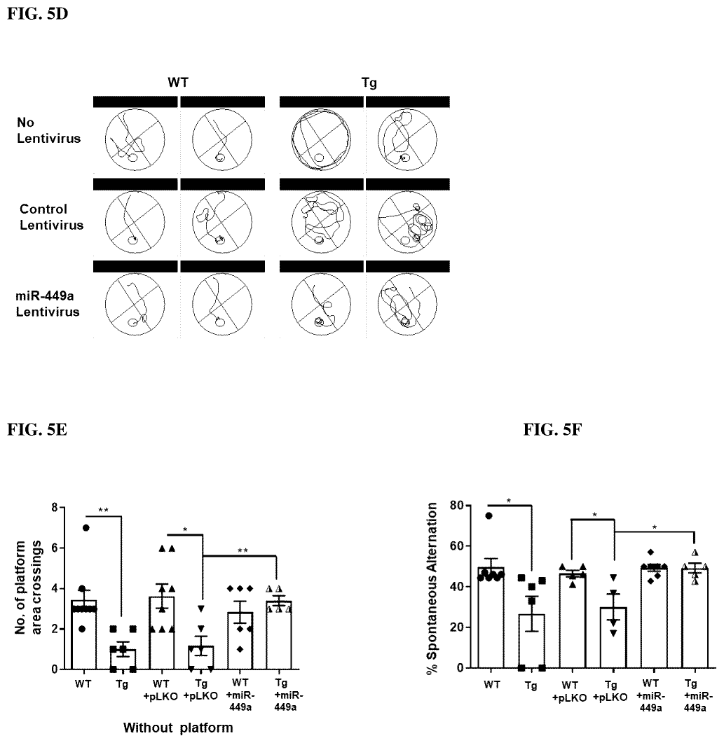

A group of six months old WT and TgAD female mice (divided in six independent cohorts) (Wang, Tanila et al. 2003) were injected bilaterally in the frontal cortex region with lentiviral particles encoding the miR-449a hairpin (Lv-miR-449a) (described above) or the negative control lentivirus (Lv-NC), which does not express any miRNA. In addition, control experiments were also performed with animals that were not injected. Subsequently, memory and learning of these animals was assessed 45 days after injection (FIG. 5A).

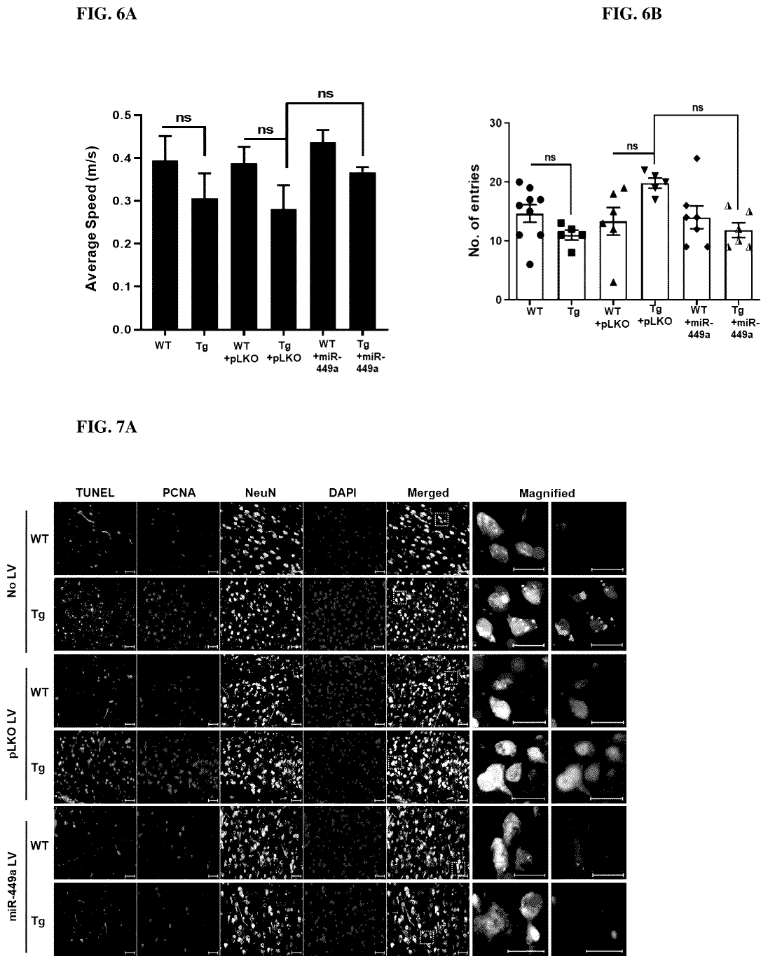

Morris water maze (MWM) test was performed to evaluate the changes in spatial learning and memory of these animals (Vorhees and Williams 2006, Nunez 2008) (FIG. 5A). We observed an increased latency in reaching the hidden platform during the training phase of TgAD mice, which was consistent with previous observations (Holcomb, Gordon et al. 1999, Chen, Chen et al. 2000, Clinton, Billings et al. 2007). Un-injected or control lentivirus (Lv-NC) injected TgAD mice showed an increased latency to reach the hidden platform relative to the corresponding WT groups. Strikingly, the TgAD mice injected with Lv-miR-449a displayed a significant decrease in the latency on the final day (day 4) of the trials (FIG. 5B, C, D). It was important to assess the stored memory, which is a frontal cortex related function (Preston and Eichenbaum 2013). Therefore, the probe trial of MWM test was in which TgAD mice exhibited decreased platform crossings than the WT animals as reported previously (Holcomb, Gordon et al. 1999, Chen, Chen et al. 2000, Clinton, Billings et al. 2007). Upon Lv-miR-449a injection TgAD animals exhibited marked improvement in the target quadrant crossings compared to TgAD groups (Un-injected or Lv-NC injected) (FIG. 5F). There was no significant change in the average swimming speed of mice in each group (FIG. 6A). That is no change in swimming speed for morris water maze test indicates that there was no difference in the physical activity of the mice and the results obtained are specifically due to cognitive abilities. These data suggested that Lv-miR-449a can improve spatial learning in TgAD mice.

The effect of the miR-449a on the working memory of TgAD mice was also evaluated by conducting the Y-maze task (FIG. 5A). The TgAD mice displayed a decrease in spontaneous alternation (Sharma, Rakoczy et al. 2010, Miedel, Patton et al. 2017) and no significant improvement was observed following control virus injections (Lv-NC) in these animals (FIG. 5F). In comparison, TgAD-Lv-miR-449a animal group showed a significant increase in the percentage of spontaneous alternations, which is an index of working memory (Goldman-Rakic 1996, Cui, Jin et al. 2011) (p<0.05, FIG. 5F). There was no significant change in number of arm entries amongst all the groups (FIG. 6B). That is no change in arm entries for Y-maze test indicates that there was no difference in the physical activity of the mice and the results obtained are specifically due to cognitive abilities. Collectively, these studies strongly indicated that miR-449a contributes to learning and memory in TgAD mice, which can be improved by the overexpression of miR-449a in TgAD mice.

### Example 7: MiR-449a Overexpression Reverts Neurodegeneration in TgAD Mice by Preventing CRNA

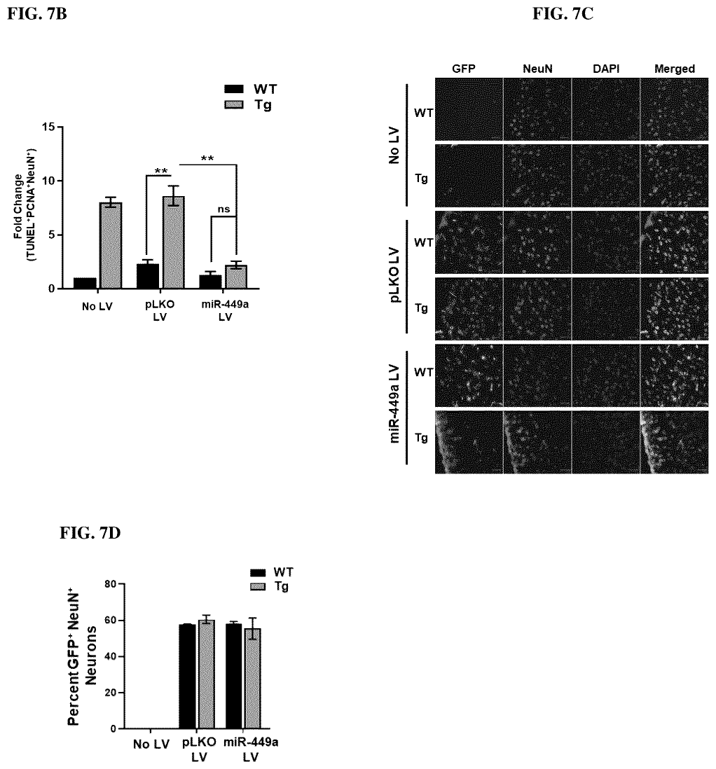

Next, the status of CRNA observed in TgAD mice upon Lv-miR-449a treatment was investigated. Therefore, the animals used in above-mentioned studies were sacrificed and their cortex were analyzed after behavioral tests. Immunohistochemistry revealed that ˜60% NeuN cortical neurons in lentivirus-injected mice exhibited GFP expression (FIG. 7C, 7D). Immunostaining for PCNA in combination with TUNEL labelling was performed on section of frontal cortex to identify neurons undergoing cell cycle re-entry (PCNA⁺) and also apoptosis (TUNEL⁺) and anti-NeuN antibody was used to specifically label neurons. Previously, aberrant cell cycle re-entry related neurodegeneration has been reported in the cortex as well as the hippocampus of AD patients and various AD animal models (Busser, Geldmacher et al. 1998, Yang, Mufson et al. 2003, Varvel, Bhaskar et al. 2008). The analysis of TgAD mice cortex revealed significantly higher PCNA⁺/TUNEL⁺ neurons in comparison to WT mice (FIG. 7A-B). Similar observations were made in control Lv-NC injected TgAD cortex. In sharp contrast, Lv-miR-449a injection resulted in a significant reduction in TUNEL⁺/PCNA⁺TgAD neurons in comparison to Lv-NC-injected counterparts (FIG. 7A-B). These data confirmed that impaired miR-449a expression can contribute to CRNA in TgAD mice, which can be overcome by overexpressing this miRNA in the cortex of these animals. Importantly, these results corroborate well with the observed reversal of defects in learning and memory in TgAD mice upon microinjection of lentivirus expressing this miRNA (FIG. 5). Collectively, these results raise the possibility of therapeutic use of miR-449a in AD.

### Behavioural Tests:

For Morris water maze test, animals were subjected to Morris Water Maze test after 45 days of injection of viral vector to examine memory and learning capacities. The test was performed for 4 days for platform tests and one probe trial (without platform). Each mouse received 3 trials (1 min each) per day for 4 consecutive days (Vorhees and Williams 2006, Nunez 2008). For each trial, mice were placed in the pool at a different start location and was allowed to swim until it either located the hidden platform or reached the end of the 60-second trial. Following completion of 4 days of testing, mice were subjected to a probe trial in which the platform was removed, and swimming behavior was monitored for 60 seconds. The learning of the platform location was evaluated by escape latency (the duration to reach the platform) during the training trials and behavior during the probe trial were measured as time spent in the platform quadrant. All events were monitored and observed in person as well as with the help of camera recordings in ANY-maze software (Stoieling Co. USA).

For Y-Maze test, mouse was randomly placed in one of the arms of the maze with one arm blocked and allowed to move for 3 min and after that the block to arm was removed and mouse was allowed to move freely for another 5 min. The series of arm entries were recorded visually and with camera setup connected of ANY-maze software. The short term memory was evaluated via spontaneous alternations as short term memory index were calculated using the following formula:

$$Percentage spotaneous alternation ( SA ) = \frac{No . ofalternations}{No . ofarmentries - 2} * 100$$

### Immunohistochemistry (IHC) and TUNEL Labelling on Brain Cryosections

After performing behavioral tests on stereotaxically injected animals, animals were deeply anesthetized and perfused with 1×PBS followed by 4% PFA. Brains were isolated and kept in 4% PFA for 12 h and then in 30% sucrose. Blocks were prepared using Leica Tissue freezing medium and stored at −80° C. 10 μm sections on poly-L-Lysine coated glass slides were prepared on a Leica cryotome.

For TUNEL labelling and IHC, slides with brain sections were twice rinsed in 1× PBS for 10 min at room temperature. Next, heat-mediated citrate buffer antigen retrieval (AR) step was performed by boiling for 10 min and gradual cooling at room temperature for 20 min and then rinsed with distilled water (Moreno-Jimenez, Flor-Garcia et al. 2019). After 1×PBS wash, 5 min proteinase K treatment was given followed by 4% PFA fixation for 3 min followed by TUNEL labeling performed by using Dead End fluorometric TUNEL system (G3250, Promega) as per manufacturer's guidelines. Subsequently, blocking was done with 3% BSA, 2% normal goat serum and 0.15% Triton X 100 for 1h followed by incubation with primary antibodies at 4° C. for 12h. To detect binding of primary antibodies, the Alexa Fluor (488 or 594) conjugated, or r-PE conjugated secondary antibodies were used. Sections were mounted with DAPI containing medium (VectaShield).

Single-plane four-channel (DAPI (blue), TUNEL (green), PCNA and NeuN (yellow)) 8-bit images (25× oil-immersion objective) were obtained with Zeiss Axiolmager1 microscope. The acquisition settings were kept constant for all images. Five fields per section and three sections per mouse were obtained for quantification of cells.

### Image and Statistical Analysis

Image J (NIH) software was used for densitometry analysis of desired bands in Western blots. The band intensity of the loading control (β-Actin) was used for the normalization. Unless indicated otherwise, one-way Analysis of Variance (ANOVA) or two tailed t-test were used for statistical analysis (Graph Pad software Inc USA). Significant results were analysed by post hoc Tukey's test. Data are represented as mean±Standard error of Mean (SEM) from three independent experiments at a 95% confidence interval, unless indicated otherwise.

### Numbered Embodiments

1. A method for expressing miR-449a in a neuronal cell, comprising introducing into the cell a viral vector comprising a nucleotide sequence containing a miRNA-449a sequence.
2. The method as recited in embodiment 1, wherein the nucleotide sequence comprises a miRNA-449a hairpin loop sequence having a mature miR-449a sequence.
3. The method as recited in embodiment 1 or 2, wherein the viral vector is a lentivirus vector.
4. The method as recited in any one of embodiments 1-3, wherein the method is an in-vivo or an in vitro method.
5. The method as recited in any of embodiments 1-4, wherein the viral vector increases the levels of miR-449a in the neuronal cell.
6. The method as recited in any one of embodiments 1-5, wherein the viral vector decreases levels of cyclin D1 and CDC25A proteins in the neuronal cell.
7. The method as recited in embodiment 6, wherein the viral vector decreases the levels of CDC25A protein in the neuronal cell by about 2-fold to about 6-fold compared to levels of CDC25A in cells transduced with a control vector.
8. The method as recited in embodiment 6, wherein the viral vector decreases the levels of cyclin D1 protein in the neuronal cell by about 2-fold to about 8-fold compared to levels of cyclin D1 in cells transduced with a control vector.
9. The method as recited in embodiment 6, wherein the viral vector decreases levels of CDC25A protein in the neuronal cell by about 30-80% percent compared to levels of CDC25A in cells transduced with a control vector.
10. The method as recited in embodiment 6, wherein the viral vector decreases levels of cyclin D1 protein in the neuronal cell by about 20-90% percent compared to levels of cyclin D1 in cells transduced with a control vector.
11. The method as recited in any one of embodiments 1-10, wherein the viral vector inhibits cell cycle related neuronal apoptosis (CRNA) by about 4 to 12-fold as measured by a decrease in levels of proliferating cell nuclear antigen (PCNA) in the neuronal cell compared to levels of PCNA in cells transduced with a control vector.
12. The method as recited in any one of embodiments 1-10, wherein the viral vector inhibits cell cycle related neuronal apoptosis (CRNA) by about 30-80% as measured by a decrease in levels of proliferating cell nuclear antigen (PCNA) in the neuronal cell compared to levels of PCNA in cells transduced with a control vector.
13. The method as recited in any one of embodiments 1-10, wherein the viral vector inhibits cell cycle related neuronal apoptosis (CRNA) by about 3 to 15-fold as measured by a decrease in levels of cleaved caspase 3 in the neuronal cell compared to levels of cleaved caspase 3 in cells transduced with a control vector.
14. The method as recited in any one of embodiments 1-10, wherein the viral vector inhibits cell cycle related neuronal apoptosis (CRNA) by about 20-90% as measured by a decrease in levels of cleaved caspase 3 in the neuronal cell when compared to levels of cleaved caspase 3 in cells transduced with a control vector.
15. A method for treating a cognitive impairment due to dysregulation of miR-449a in a patient in need thereof, comprising administering to the patient a viral vector comprising a nucleotide sequence containing a miRNA-449a sequence.
16. The method of embodiment 15, wherein the miRNA-449a sequence comprises a miRNA-449a hairpin loop sequence including mature miR-449a sequence.
17. The method of embodiment 15 or 16, wherein the viral vector is administered intramuscularly, intravenously, intrathecally, intraparenchymally or intracerebroventricularly.
18. The method of any one of embodiments 15-17, wherein the viral vector is a lentivirus vector.
19. The method of any one of embodiments 15-18, wherein the administration of the viral vector increases levels of miR-449a in neuronal cells of the patient.
20. The method of any one of embodiments 15-19, wherein the administration of the viral vector decreases levels of cyclin D1 and CDC25A proteins in neuronal cells of the patient.
21. The method of embodiment 20, wherein the administration of the viral vector to the patient decreases the levels of CDC25A protein in neuronal cells of the patient by about 2-fold to about 6-fold compared to levels of CDC25A prior to administration of the viral vector.
22. The method of embodiment 20, wherein the administration of the viral vector to the patient decreases the levels of cyclin D1 protein in neuronal cells of the patient by about 2-fold to about 8-fold compared to levels of cyclin D1 prior to administration of the viral vector.
23. The method of embodiment 20, wherein the administration of the viral vector to the patient decreases levels of CDC25A protein in neuronal cells of the patient by about 30-80% percent compared to levels of CDC25A prior to administration of the viral vector.
24. The method of embodiment 20, wherein administration of the viral vector to the patient decreases levels of cyclin D1 protein in neuronal cells of the patient by about 20-90% percent compared to levels of cyclin D1 prior to administration of the viral vector.
25. The method of any one of embodiments 15-24, wherein administration of the viral vector to the patient inhibits cell cycle related neuronal apoptosis (CRNA) in the patient by about 4 to 12-fold as measured by a decrease in levels of proliferating cell nuclear antigen (PCNA) when compared to levels of PCNA prior to administration of the viral vector.
26. The method of any one of embodiments 15-24, wherein administration of the viral vector to the patient inhibits cell cycle related neuronal apoptosis (CRNA) in the patient by about 30-80% as measured by a decrease in levels of proliferating cell nuclear antigen (PCNA) when compared to levels of PCNA prior to administration of the viral vector.
27. The method of any one of embodiments 15-24, wherein administration of the viral vector to the patient inhibits cell cycle related neuronal apoptosis (CRNA) in the patient by about 3 to 15-fold as measured by a decrease in levels of cleaved caspase 3 when compared to levels of cleaved caspase 3 prior to administration of the viral vector.
28. The method of any one of embodiments 15-24, wherein administration of the viral vector to the patient inhibits cell cycle related neuronal apoptosis (CRNA) in the patient by about 20-90% as measured by a decrease in levels of cleaved caspase 3 when compared to levels of cleaved caspase 3 prior to administration of the viral vector.
29. The method of any one of embodiments 15-28, wherein administration of the viral vector to the patient improves short-term and long-term memory in the patient.
30. The method of any one of embodiments 15-29, wherein administration of the viral vector to the patient improves a learning ability of the patient.
31. The method of any one of embodiments 15-30, wherein the cognitive impairment due to miR-449a dysregulation is associated with a degenerative neurological disorder.
32. The method of embodiment 31, wherein the degenerative neurological disorder is Alzheimer's disease.
33. A composition comprising a viral vector comprising a nucleotide sequence containing miR-449a sequence for use in treating a cognitive impairment due to miR-449a dysregulation.
34. The composition for use as recited in embodiment 33, wherein the miR-449a sequence comprises a miRNA-449 hairpin loop sequence including mature miR-449a sequence.
35. The composition for use as recited in embodiment 33 or 34, wherein the administration of the composition increases levels of miR-449a in neuronal cells of the patient.
36. The composition for use as recited in any one of embodiments 33-35, wherein the composition is in an injection or an infusion form.
37. The composition for use as recited in any one of embodiments 33-36, wherein the composition decreases the levels of CDC25A protein in neuronal cells of the patient by about 2 to about 6-fold compared to levels of CDC25A prior to administration of the composition.
38. The composition for use as recited in any one of embodiments 33-36, wherein the composition decreases levels of cyclin D1 in neuronal cells of the patient by about 2 to about 8-fold compared to levels of cyclin D1 prior to administration of the viral vector.
39. The composition for use as recited in any one of embodiments 33-36, wherein the composition decreases levels of CDC25A protein in neuronal cells of the patient by about 30-80% percent compared to levels of CDC25A prior to administration of the composition.
40. The composition for use as recited in any one of embodiments 33-36, wherein the composition decreases levels of cyclin D1 protein in neuronal cells of the patient by about 20-90% percent compared to levels of cyclin D1 prior to administration of the viral vector.
41. The composition for use as recited in any one of embodiments 33-36, wherein the composition inhibits cell cycle related neuronal apoptosis (CRNA) in the patient by about 4 to 12-fold as measured by a decrease in levels of proliferating cell nuclear antigen (PCNA) when compared to levels of PCNA prior to administration of the composition.
42. The composition for use as recited in any one of embodiments 33-36, wherein the composition inhibits cell cycle related neuronal apoptosis (CRNA) in the patient by about 30-80% as measured by a decrease in levels of proliferating cell nuclear antigen (PCNA) when compared to levels of PCNA prior to administration of the composition.
43. The composition for use as recited in any one of embodiments 33-36, wherein the composition inhibits cell cycle related neuronal apoptosis (CRNA) in a patient by about 3 to 15-fold as measured by a decrease in levels of cleaved caspase 3 when compared to levels of cleaved caspase 3 prior to administration of the composition.
44. The composition for use as recited in any one of embodiments 33-36, wherein the composition inhibits cell cycle related neuronal apoptosis (CRNA) in the patient by about 20-90% as measured by a decrease in levels of cleaved caspase 3 when compared to levels of cleaved caspase 3 prior to administration of the composition.
45. The composition for use as recited in any one of embodiments 33-44, wherein the composition improves short-term and long-term memory in a patient.
46. The composition for use as recited in any one of embodiments 33-45, wherein the composition improves a learning ability of the patient.
47. The composition for use as recited in any one of embodiments 33-46, wherein the cognitive impairment due to miR-449a dysregulation is associated with a degenerative neurological disorder.
48. The composition for use as recited in embodiment 47, wherein the degenerative neurological disorder is Alzheimer's disease.
49. Use of a viral vector comprising a nucleotide sequence containing a miRNA-449a sequence in the manufacture of a medicament for treating a cognitive impairment due to dysregulation of miR-449a.
50. The use as recited in embodiment 49, wherein the miRNA-449a sequence comprises a miRNA-449 hairpin loop sequence including mature miRNA-449a sequence.
51. The use as recited in embodiment 49 or 50, wherein the viral vector is a lentivirus vector.
52. The use as recited in any one of embodiments 49-51, wherein the medicament is in an injection or an infusion form.

1.-16. (canceled)

17. A method for treating a cognitive impairment due to dysregulation of miR-449a in a patient in need thereof, comprising administering to the patient a viral vector comprising a nucleotide sequence containing a miRNA-449a sequence.

18. The method of claim 17, wherein the miRNA-449a sequence comprises a miRNA-449a hairpin loop sequence including mature miR-449a sequence.

19. The method of claim 17, wherein the viral vector is administered intramuscularly, intravenously, intrathecally, intraparenchymally or intracerebroventricularly.

20. The method of claim 17, wherein the viral vector is a lentivirus vector.

21. The method of claim 17, wherein the administration of the viral vector increases levels of miR-449a in neuronal cells of the patient.

22. The method of claim 17, wherein the administration of the viral vector decreases levels of cyclin D1 and CDC25A proteins in neuronal cells of the patient.

23. The method of claim 22, wherein the administration of the viral vector to the patient decreases the levels of CDC25A protein in neuronal cells of the patient by about 2-fold to about 6-fold compared to levels of CDC25A prior to administration of the viral vector.

24. The method of claim 22, wherein the administration of the viral vector to the patient decreases the levels of cyclin D1 protein in neuronal cells of the patient by about 2-fold to about 8-fold compared to levels of cyclin D1 prior to administration of the viral vector.

25. The method of claim 22, wherein the administration of the viral vector to the patient decreases levels of CDC25A protein in neuronal cells of the patient by about 30-80% percent compared to levels of CDC25A prior to administration of the viral vector.

26. The method of claim 22, wherein administration of the viral vector to the patient decreases levels of cyclin D1 protein in neuronal cells of the patient by about 20-90% percent compared to levels of cyclin D1 prior to administration of the viral vector.

27. The method of claim 17, wherein administration of the viral vector to the patient inhibits cell cycle related neuronal apoptosis (CRNA) in the patient by about 4 to 12-fold as measured by a decrease in levels of proliferating cell nuclear antigen (PCNA) when compared to levels of PCNA prior to administration of the viral vector.

28. The method of claim 17, wherein administration of the viral vector to the patient inhibits cell cycle related neuronal apoptosis (CRNA) in the patient by about 30-80% as measured by a decrease in levels of proliferating cell nuclear antigen (PCNA) when compared to levels of PCNA prior to administration of the viral vector.

29. The method of claim 17, wherein administration of the viral vector to the patient inhibits cell cycle related neuronal apoptosis (CRNA) in the patient by about 3 to 15-fold as measured by a decrease in levels of cleaved caspase 3 when compared to levels of cleaved caspase 3 prior to administration of the viral vector.

30. The method of claim 17, wherein administration of the viral vector to the patient inhibits cell cycle related neuronal apoptosis (CRNA) in the patient by about 20-90% as measured by a decrease in levels of cleaved caspase 3 when compared to levels of cleaved caspase 3 prior to administration of the viral vector.

31. The method of claim 17, wherein administration of the viral vector to the patient improves short-term and long-term memory in the patient.

32. The method of claim 17, wherein administration of the viral vector to the patient improves a learning ability of the patient.

33. The method of claim 17, wherein the cognitive impairment due to miR-449a dysregulation is associated with a degenerative neurological disorder.

34. The method of claim 33, wherein the degenerative neurological disorder is Alzheimer's disease.

35.-52. (canceled)
